(19) United States
(12) Patent Application Publication
(10) Pub. No.: US 2007/0213690 A1
(43) Pub. Date: Sep. 13, 2007

(54) Blood conduit connector

(76) Inventors: Nickolas Phillips, Aliso Viejo, CA (US); Anthony Viole, Foothil Ranch, CA (US); Michael Scott, Lake Forest, CA (US); Robert Pecor, Aliso Viejo, CA (US); Shawn D. O'Leary, Mission Viejo, CA (US); Dominic K. Dinh, Rancho Santa Margarita, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/371,208

(22) Filed: Mar. 8, 2006

(51) Int. Cl. A61M 39/10; A61M 37/00; F16L 33/00

(52) U.S. Cl. 604/533; 604/131; 285/248

(57) ABSTRACT

A conduit for use with a blood pump can have a flared inner surface at an end of the conduit. In some embodiments, the conduit can include a strain relief member such as overmolded silicone. The strain relief member can maintain the profile of the flared inner surface. A conduit with a flared inner surface can be configured for connection to a blood pump via a connector. For example, the conduit can include an anchor member such as flange configured to engage with various components of a connector to connect the conduit to a fitting on a pump or another structure. In some embodiments, the conduit can be configured to connect to a blood conduit connector including a member such as a compression collet and a fitting such as a coupler. The connector can include a bayonet connection to facilitate rapid connection and disconnection of the conduit from a fitting.

### BACKGROUND OF THE INVENTION

1. Field of the Invention

This application relates generally to connectors for fluid flow conduits, which can be used to couple a blood flow conduit to a blood pump in a blood flow system.

2. Description of the Related Art

Dialysis and other medical procedures have been implemented to treat blood in patients. In dialysis, blood is removed from and then returned to the patient after being treated. The treatment can, for example, remove impurities from the blood, a function performed by the kidney in a healthy person. Typically, blood is withdrawn via a first catheter, forced through a filter, and returned to the patient via a second catheter. Blood flow systems such as pumping systems to enhance or support circulatory function can similarly withdraw blood with a first catheter, and return blood to the patient via pump with a second catheter.

Various techniques have been developed to apply these systems in a manner that allows connection of a tube to pump or filter. For example, a tube can be forced over a port, where the tube and port are the same size. The connection requires the tube to be deformed be advanced over the ports. As such, the connection therebetween is cumbersome, and can result in damage to the tube, possibly weakening the tube to a point where the tube may fall.

### SUMMARY OF THE INVENTION

It would be advantageous to have devices and techniques that enable quickly connecting two fluid conveying portions of a fluid circuit. Such connecting would enable the two fluid conveying portions to be connected together whereby the risk of introduction of embolic matter or material, e.g., gas, is reduced or eliminated. Preferably such system will be easy to use and will result in minimum spillage of fluids.

In certain embodiments, an apparatus is disclosed. The apparatus comprises a connector fitting, a conduit, a member, and a coupler. The connector fitting has a distal end, a blood flow lumen, and an outer surface. The conduit comprises a biocompatible material. The conduit has a pre-formed flared proximal portion. The member is configured to be disposed around and to extend along at least a portion of the proximal portion of the conduit. The coupler is configured to be urged over the member and the conduit proximally relative to the connector fitting to apply pressure to the conduit to secure the conduit to the connector fitting. In other embodiments, a blood flow system is provided. The blood flow system comprises a pump, a conduit, and a coupler. The pump has a connector fitting comprising a blood flow lumen. The conduit is constructed of a biocompatible material. The conduit has a proximal portion that is flared in its free state. The conduit has a strain relief member disposed over the flared proximal portion. The coupler is configured to be urged over the conduit proximally relative to the connector fitting to apply pressure to the conduit to secure the conduit to the connector fitting.

In other embodiments, a method of establishing a connection between a conduit and a connector fitting extending from a pump inlet port or a pump outlet port is provided. The method comprises the steps of advancing a conduit having a pre-flared portion toward the connector fitting; urging a coupling device over the pre-flared portion of the conduit; and engaging the coupling device with the connector fitting.

In other embodiments, a conduit for use with a blood pump is provided. The conduit comprises a biocompatible material. The conduit has a flared inner surface at one end thereof. The conduit is configured to mechanically engage a connector

### BRIEF DESCRIPTION OF THE DRAWINGS

These and other features and advantages of the invention will now be described with reference to the drawings, which are intended to illustrate and not to limit the invention.

FIG. 1 is a schematic view of one embodiment of a heart assist system having multiple conduits for multi-site application, shown applied to a patient's vascular system;

FIG. 2 is a schematic view of another application of the embodiment of FIG. 1;

FIG. 3 is a schematic view of another embodiment of a heart assist system having multiple conduits for multi-site application wherein each of the conduits is applied to more than one vessel, shown applied to a patient's vascular system;

FIG. 4 is a schematic view of another embodiment of a heart assist system having multiple conduits for multi-site application and employing a connector with a T-shaped fitting, shown applied to a patient's vascular system;

FIG. 5 is a schematic view of an L-shaped connector coupled with an inflow conduit, shown inserted within a blood vessel;

FIG. 6 is a schematic view of another embodiment of a heart assist system having multiple conduits for multi-site application, shown applied to a patient's vascular system;

FIG. 7 is a schematic view of another application of the embodiment of FIG. 6, shown applied to a patient's vascular system;

FIG. 8 is a schematic view of another application of the embodiment of FIG. 6, shown applied to a patient's vascular system;

FIG. 9 is a schematic view of another embodiment of a heart assist system having multiple conduits for multi-site application, a reservoir, and a portable housing for carrying a portion of the system directly on the patient;

FIG. 10 is a schematic view of another embodiment of a heart assist system having a multilumen cannula for single-site application, shown applied to a patient's vascular system;

FIG. 11 is a schematic view of a modified embodiment of the heart assist system of FIG. 10, shown applied to a patient's vascular system;

FIG. 12 is a schematic view of another embodiment of a heart assist system having multiple conduits for single-site application, shown applied to a patient's circulatory system;

FIG. 13 is a schematic view of another application of the embodiment of FIG. 12, shown applied to a patient's vascular system;

FIG. 14 is a schematic view of one application of an embodiment of a heart assist system having an intravascular pump enclosed in a protective housing, wherein the intravascular pump is inserted into the patient's vasculature through a non-primary vessel;

FIG. 15 is a schematic view of another embodiment of a heart assist system having an intravascular pump housed within a conduit having an inlet and an outlet, wherein the intravascular pump is inserted into the patient's vasculature through a non-primary vessel;

FIG. 16 is a schematic view of a modified embodiment of the heart assist system of FIG. 15 in which an additional conduit is shown adjacent the conduit housing the pump, and in which the pump comprises a shaft-mounted helical thread;

FIG. 17 is a perspective view of one embodiment of a blood conduit connector applicator assembly;

FIG. 18 is an exploded perspective view of the blood conduit connector applicator assembly of FIG. 17;

FIG. 19 is a perspective view of one embodiment of a blood conduit connector assembly;

FIG. 19A is a longitudinal cross-sectional view of FIG. 19 taken through section plane 19A-19A;

FIG. 19B is a detail view of the cross-sectional view of FIG. 19A;

FIG. 20 is an exploded perspective view of the blood conduit connector assembly of FIG. 19;

FIG. 21 is pump-side or proximal end perspective view of one embodiment of a pump fitting;

FIG. 22 is graft-side or distal end perspective view of the pump fitting of FIG. 21;

FIG. 23 is a pump-end view of the pump fitting of FIG. 21;

FIG. 24 is a cross-sectional view of the pump fitting of FIG. 21 taken through section plane 24-24;

FIG. 25 is a detail view of a portion of the graft end of the pump fitting taken at line 25-25;

FIG. 26 is a graft-end view of the pump fitting of FIG. 21;

FIG. 27 is a plan view of the pump fitting of FIG. 21;

FIG. 28 is a detail view of a coupler engagement portion taken at line 28-28;

FIG. 29 is a perspective view of one embodiment of a graft assembly comprising a flared portion;

FIG. 30 is a plan view of the graft assembly of FIG. 29;

FIG. 31 is an end view of the graft assembly of FIG. 29;

FIG. 32 is a perspective view of one embodiment of a vascular graft that can be incorporated into the graft assembly of FIG. 29;

FIG. 33 is a plan view of the vascular graft of FIG. 32;

FIG. 34 is an end view of the vascular graft assembly of FIG. 32;

FIG. 35 is a perspective view of one embodiment of a compression collet;

FIG. 36 is a vessel or distal end view of the compression collet of FIG. 35;

FIG. 37 is a cross-sectional view of the compression collet of FIG. 36 taken at section 37-37 shown in FIG. 36;

FIG. 38 is a perspective view taken from a distal end of one embodiment of a coupler;

FIG. 39 is a perspective view taken from a proximal end of the coupler of FIG. 38;

FIG. 40 is a plan view of the coupler of FIG. 38;

FIG. 41 is a distal end view of the coupler of FIG. 38;

FIG. 42 is a cross-sectional view of the coupler of FIG. 38 taken at section plane 42-42 shown in FIG. 41;

FIG. 43 is a proximal end view of the coupler of FIG. 38;

FIG. 44 is a cross-sectional view of the coupler of FIG. 38 taken at section plane 44-44 shown in FIG. 43;

FIG. 45 is a perspective view of one embodiment of an applicator tool that can be used to apply to or remove a blood conduit connector assembly from another component of a system configured to convey blood;

FIG. 46 is an end view of the applicator tool of FIG. 45;

FIG. 47 is a cross-sectional view of the applicator tool of FIG. 45 at section plane 47-47 shown in FIG. 46;

FIG. 48 is an end view of the applicator tool of FIG. 45;

FIG. 49 is a cross-sectional view of the applicator tool of FIG. 45 at section plane 49-49 shown in FIG. 48.

### DETAILED DESCRIPTION OF THE PREFERRED EMBODIMENT

This application is directed to apparatuses, systems, and methods for coupling a blood conduit with the vasculature of a patient. The coupling or connection between the blood conduit and the vasculature can be achieved by any suitable means or technique and can be for any purpose. One application or treatment with which the coupling or connection is useful is in connection with a blood supplementation system, and particularly in connection with such a system that is configured for implantation within a patient. Such an implantable system is particularly useful for long-term application or use. As discussed further below, various embodiments of blood conduit connector applicator assemblies and blood conduit connector assemblies are particularly advantageous.

In one aspect, a blood conduit connector assembly comprises a connector device that can be used in an implantable blood supplementation system. Such a system can be configured to circulate blood between two vascular locations through a pump and two blood flow conduits. The pump can be implantable. One or more of the conduits can be graft cannula(e) fluidly coupled with, e.g., physically connected to the vasaculature. The conduits can take other forms, as discussed below. The conduits or grafts can be coupled with the vasculature at two different vascular locations that can be spaced apart by a suitable amount. In such a system, the blood conduit connector assemblies, connection devices, and connectors can be used to provide a secure connection between the pump and a cannula, e.g., a graft. Various embodiments of systems with which the system can be used are discussed herein.

Turning now to the drawings provided herein, more detailed descriptions of various embodiments of heart assist systems and cannulae for use therewith are provided below.

### I. Extracardiac Heart Assist Systems and Methods

A variety of cannulae and cannula assemblies are described herein that can be used in connection with a variety of heart assist systems that supplement perfusion. Such systems preferably are extracardiac in nature. In other words, the systems supplement blood perfusion, without the need to interface directly with the heart and aorta. Thus, the systems can be applied without major invasive surgery. The systems also lessen the hemodynamic burden or workload on the heart by reducing afterload, impedence, and/or left ventricular end diastolic pressure and volume (preload). The systems also advantageously increase peripheral organ perfusion and provide improvement in neurohormonal status. As discussed more fully below, the systems can be applied using one or more cannulae, one or more vascular grafts, and a combination of one or more cannulae and one or more vascular grafts. For systems employing cannula(e), the cannula(e) can be applied through multiple percutaneous insertion sites (sometimes referred to herein as a multi-site application) or through a single percutaneous insertion site (sometimes referred to herein as a single-site application).

A. Heart Assist Systems and Methods Employing Multi-site Application

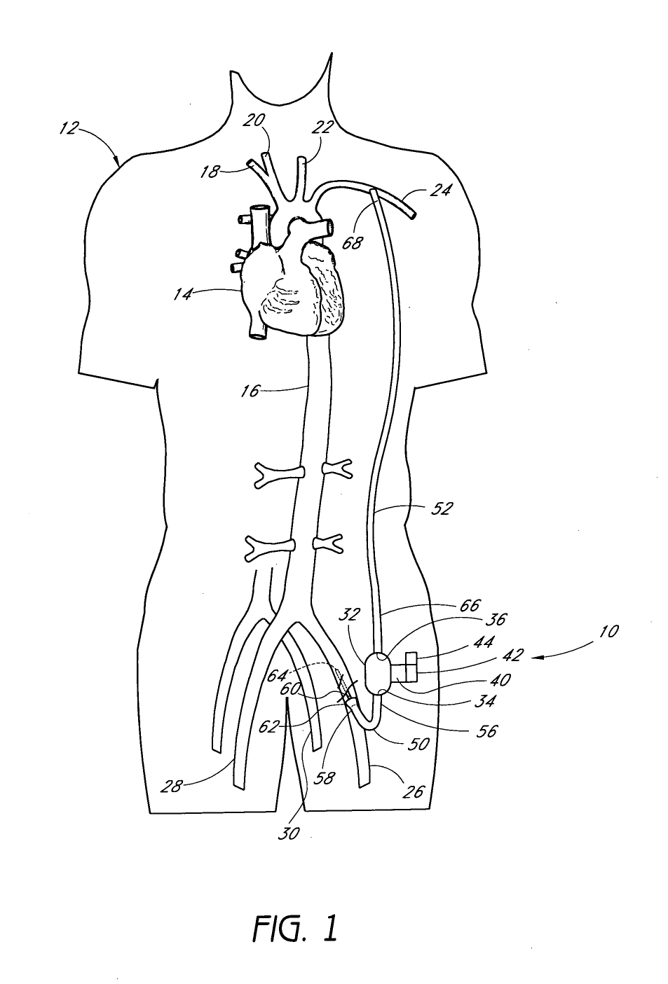

With reference to FIG. 1, a first embodiment of a heart assist system 10 is shown applied to a patient 12 having an ailing heart 14 and an aorta 16, from which peripheral brachiocephalic blood vessels extend, including the right subclavian artery 18, the right carotid artery 20, the left carotid artery 22, and the left subclavian artery 24. Extending from the descending aorta is another set of peripheral blood vessels, the left and right iliac arteries which transition into the left and right femoral arteries 26, 28, respectively. As is known, each of the arteries 16, 18, 20, 22, 24, 26, and 28 generally conveys blood away from the heart. The vasculature includes a venous system that generally conveys blood to the heart. As will be discussed in more detail below, the heart assist systems described herein can also be applied to non-primary veins, including the left femoral vein 30.

The heart assist system 10 comprises a pump 32, having an inlet 34 and an outlet 36 for connection of conduits thereto. The pump 32 preferably is a rotary pump, either an axial type or a centrifugal type, although other types of pumps may be used, whether commercially-available or customized. The pump 32 preferably is sufficiently small to be implanted subcutaneously and preferably extrathoracically, for example in the groin area of the patient 12, without the need for major invasive surgery. Because the heart assist system 10 is an extracardiac system, no valves are necessary. Any inadvertent backflow through the pump 32 and/or through the inflow conduit would not harm the patient 12.

Regardless of the style or nature chosen, the pump 32 is sized to generate blood flow at subcardiac volumetric rates, less than about 50% of the flow rate of an average healthy heart, although flow rates above that may be effective. Thus, the pump 32 is sized and configured to discharge blood at volumetric flow rates anywhere in the range of 0.1 to 3 liters per minute, depending upon the application desired and/or the degree of need for heart assist. For example, for a patient experiencing advanced congestive heart failure, it may be preferable to employ a pump that has an average subcardiac rate of 2.5 to 3 liters per minute. In other patients, particularly those with minimal levels of heart failure, it may be preferable to employ a pump that has an average subcardiac rate of 0.5 liters per minute or less. In yet other patients it may be preferable to employ a pump that is a pressure wave generator that uses pressure to augment the flow of blood generated by the heart.

In one embodiment, the pump 32 is a continuous flow pump which superimposes continuous blood-flow on the pulsatile aortic blood-flow. In another embodiment, the pump 32 has the capability of synchronous actuation; i.e., it may be actuated in a pulsatile mode, either in copulsating or counterpulsating fashion.

For copulsating action, it is contemplated that the pump 32 would be actuated to discharge blood generally during systole, beginning actuation, for example, during isovolumic contraction before the aortic valve opens or as the aortic valve opens. The pump 32 would be static while the aortic valve is closed following systole, ceasing actuation, for example, when the aortic valve closes.

For counterpulsating actuation, it is contemplated that the pump 32 would be actuated generally during diastole, ceasing actuation, for example, before or during isovolumic contraction. Such an application would permit and/or enhance coronary blood perfusion. In this application, it is contemplated that the pump 32 would be static during the balance of systole after the aortic valve is opened, to lessen the burden against which the heart must pump. The aortic valve being open encompasses the periods of opening and closing, wherein blood is flowing therethrough.

It should be recognized that the designations copulsating and counterpulsating are general identifiers and are not limited to specific points in the patient's heart cycle when the pump 32 begins and discontinues actuation. Rather, they are intended to generally refer to pump actuation in which the pump 32 is actuating, at least in part, during systole and diastole, respectively. For example, it is contemplated that the pump 32 might be activated to be out of phase from true copulsating or counterpulsating actuation described herein, and still be synchronous, depending upon the specific needs of the patient or the desired outcome. One might shift actuation of the pump 32 to begin prior to or after isovolumic contraction or to begin before or after isovolumic relaxation.

Furthermore, the pulsatile pump may be actuated to pulsate asynchronously with the patient's heart. Typically, where the patient's heart is beating irregularly, there may be a desire to pulsate the pump 32 asynchronously so that the perfusion of blood by the heart assist system 10 is more regular and, thus, more effective at oxygenating the organs. Where the patient's heart beats regularly, but weakly, synchronous pulsation of the pump 32 may be preferred.

The pump 32 is driven by a motor 40 and/or other type of drive means and is controlled preferably by a programmable controller 42 that is capable of actuating the pump 32 in pulsatile fashion, where desired, and also of controlling the speed or output of the pump 32. For synchronous control, the patient's heart would preferably be monitored with an EKG in which feedback would be provided the controller 42. The controller 42 is preferably programmed by the use of external means. This may be accomplished, for example, using RF telemetry circuits of the type commonly used within implantable pacemakers and defibrillators. The controller may also be autoregulating to permit automatic regulation of the speed, and/or regulation of the synchronous or asynchronous pulsation of the pump 32, based upon feedback from ambient sensors monitoring parameters, such as pressure or the patient's EKG. It is also contemplated that a reverse-direction pump be utilized, if desired, in which the controller is capable of reversing the direction of either the drive means or the impellers of the pump. Such a pump might be used where it is desirable to have the option of reversing the direction of circulation between two blood vessels.

Power to the motor 40 and the controller 42 may be provided by a power source 44, such as a battery, that is preferably rechargeable by an external induction source (not shown), such as an RF induction coil that may be electromagnetically coupled to the battery to induce a charge therein. Alternative power sources are also possible, including a device that draws energy directly from the patient's body; e.g., the patient's muscles, chemicals or heat. The pump can be temporarily stopped during recharging with no appreciable life threatening effect, because the system only supplements the heart, rather than substituting for the heart.

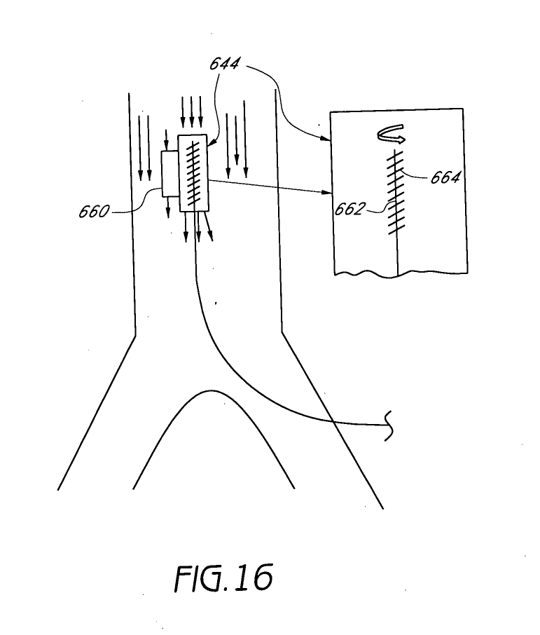

While the controller 42 and power source 44 are preferably pre-assembled to the pump 32 and implanted therewith, it is also contemplated that the pump 32 and motor 40 be implanted at one location and the controller 42 and the power source 44 be implanted in a separate location. In one alternative arrangement, the pump 32 may be driven externally through a percutaneous drive line or cable, as shown in FIG. 16. In another variation, the pump, motor and controller may be implanted and powered by an extracorporeal power source. In the latter case, the power source could be attached to the side of the patient to permit fully ambulatory movement.

The inlet 34 of the pump 32 is preferably connected to an inflow conduit 50 and an outflow conduit 52 to direct blood flow from one peripheral blood vessel to another. The conduits 50, 52 preferably are flexible conduits, as discussed more fully below. The conduits 50, 52 are coupled with the peripheral vessels in different ways in various embodiments of the heart assist system 10. As discussed more fully below, at least one of the conduits 50, 52 can be connected to a peripheral vessel, e.g., as a graft, using an anastomosis connection, and at least one of the conduits 50, 52 can be coupled with the same or another vessel via insertion of a cannula into the vasculature. Also, more than two conduits are used in some embodiments, as discussed below.

The inflow and outflow conduits 50, 52 may be formed from Dacron, Hemashield, Gortex, PVC, polyurethane, PTFE, ePTFE, nylon, or PEBAX materials, although other synthetic materials may be suitable. The inflow and outflow conduits 50, 52 may also comprise biologic materials or pseudobiological (hybrid) materials (e.g., biologic tissue supported on a synthetic scaffold). The inflow and outflow conduits 50, 52 are preferably configured to minimize kinks so blood flow is not meaningfully interrupted by normal movements of the patient or compressed easily from external forces. In some cases, the inflow and/or outflow conduits 50, 52 may come commercially already attached to the pump 32. Where it is desired to implant the pump 32 and the conduits 50, 52, it is preferable that the inner diameter of the conduits 50, 52 be less than 25 mm, although diameters slightly larger may be effective.

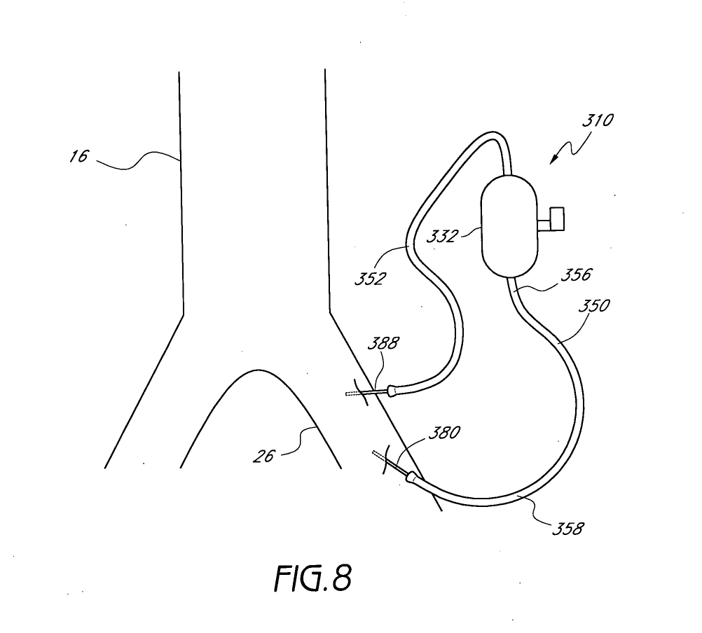

In one preferred application, the heart assist system 10 is applied in an arterial-arterial fashion; for example, as a femoral-axillary connection, as is shown in FIG. 1. It should be appreciated by one of ordinary skill in the art that an axillary-femoral connection would also be effective using the embodiments described herein. Indeed, it should be recognized by one of ordinary skill in the art that the present invention might be applied to any of the peripheral blood vessels in the patient. Another application of the heart assist system 10 couples the conduits 50, 52 with the same non-primary vessel in a manner similar to the application shown in FIG. 8 and discussed below.

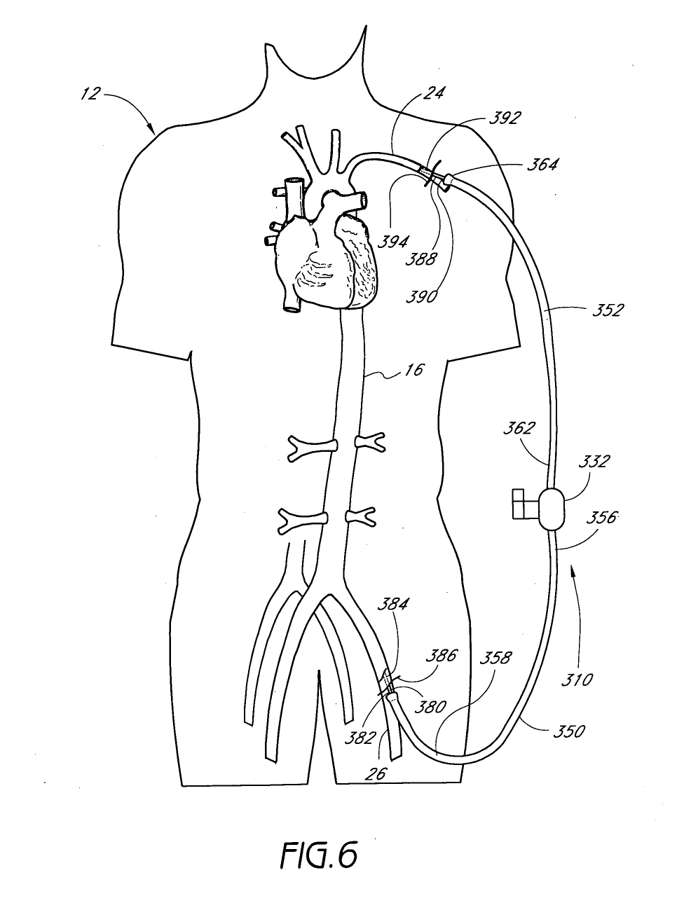

FIG. 1 shows that the inflow conduit 50 has a first end 56 that connects with the inlet 34 of the pump 32 and a second end 58 that is coupled with a first non-primary blood vessel (e.g., the left femoral artery 26) by way of an inflow cannula 60. The inflow cannula 60 has a first end 62 and a second end 64. The first end 62 is sealably connected to the second end 58 of the inflow conduit 50. The second end 64 is inserted into the blood vessel (e.g., the left femoral artery 26). Although shown as discrete structures in FIG. 1, one skilled in the art would recognize that the inflow conduit 50 and the cannula 60 may be unitary in construction.

Where the conduit 50 is at least partially extracorporeal, the inflow cannula 60 also may be inserted through a surgical opening (e.g., as shown in FIG. 6 and described in connection therewith) or percutaneously, with or without an introducer sheath (not shown). In other applications, the inflow cannula 60 could be inserted into the right femoral artery or any other peripheral artery.

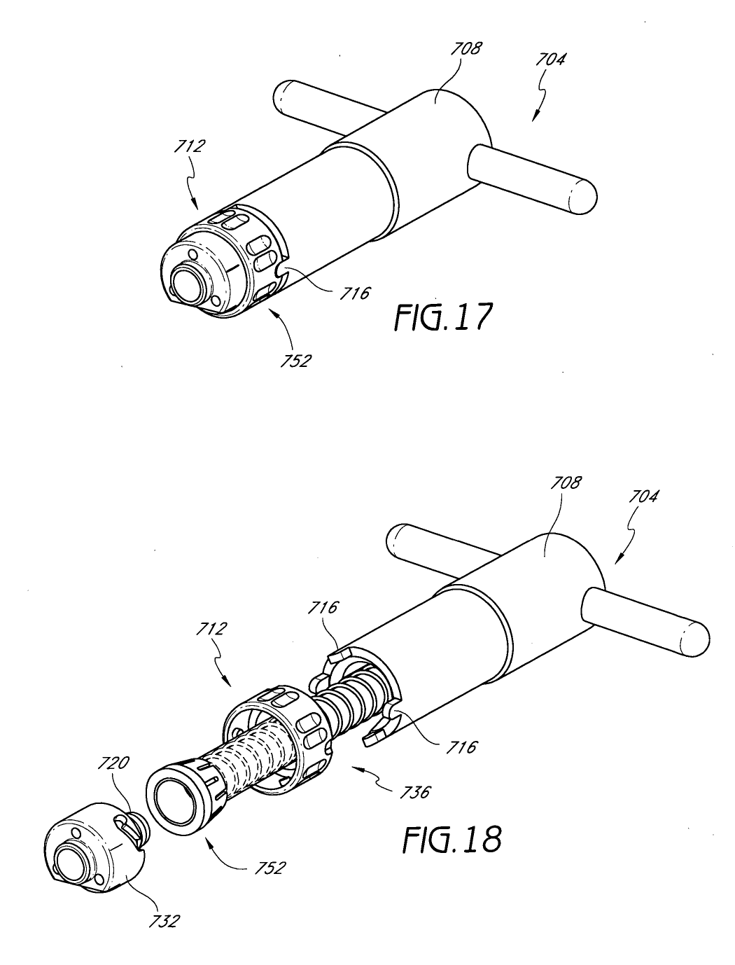

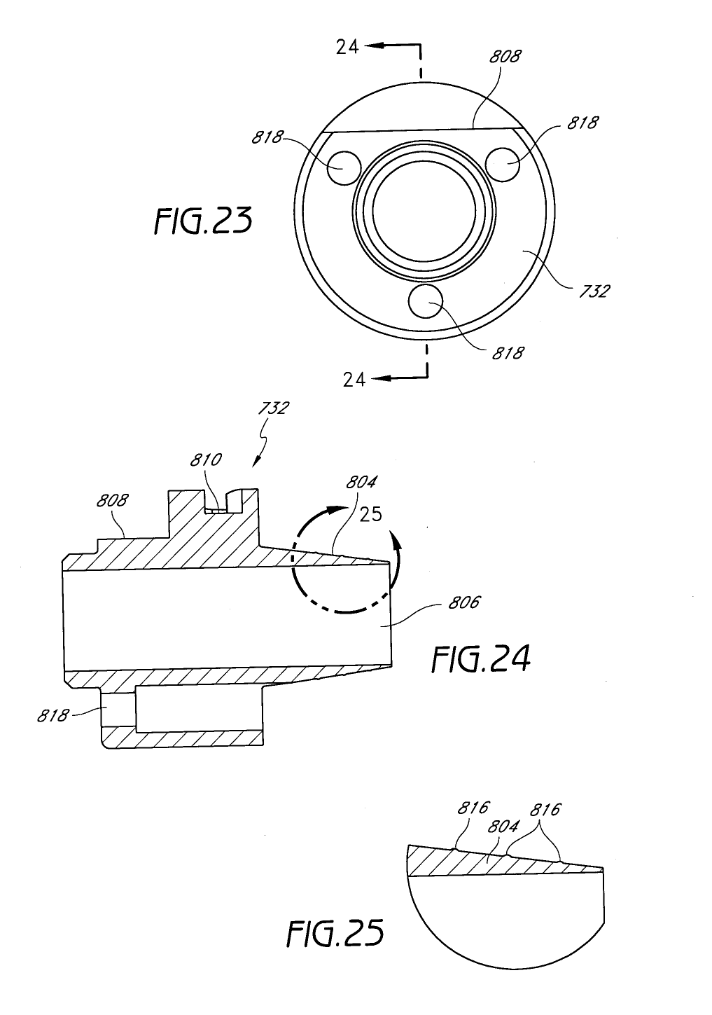

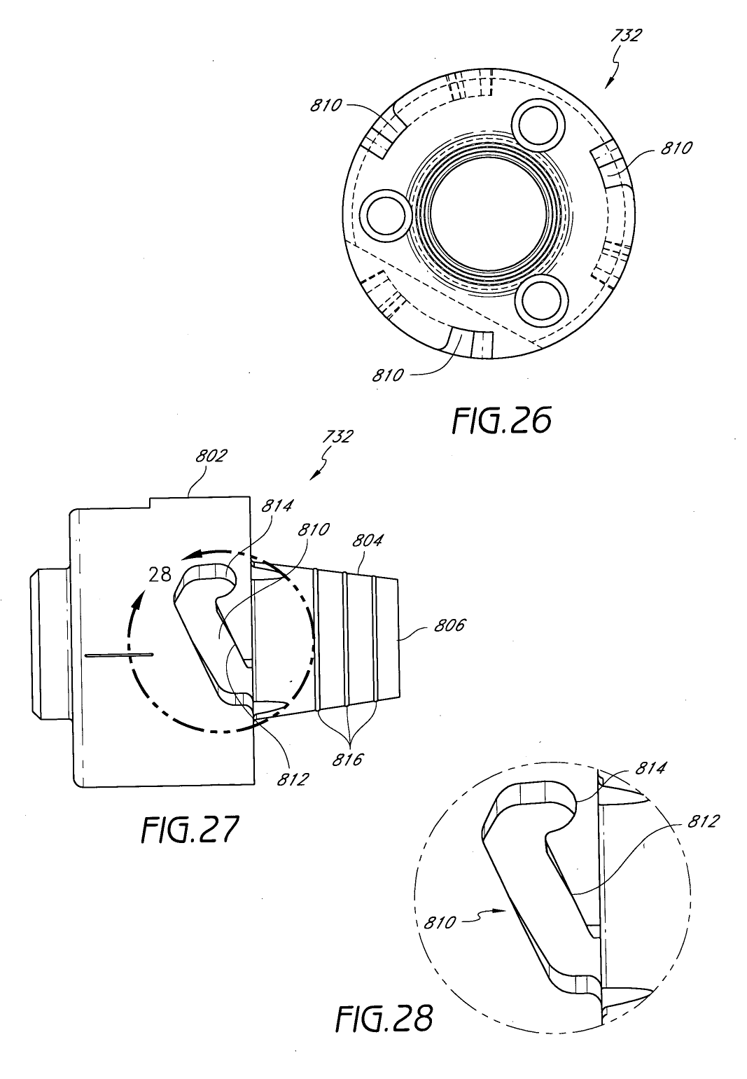

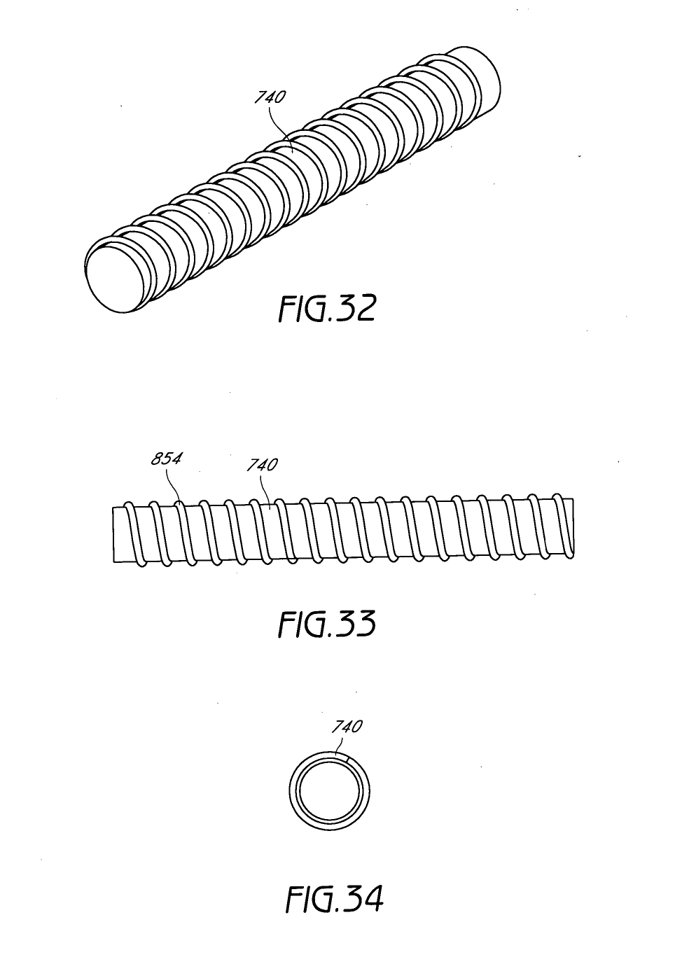

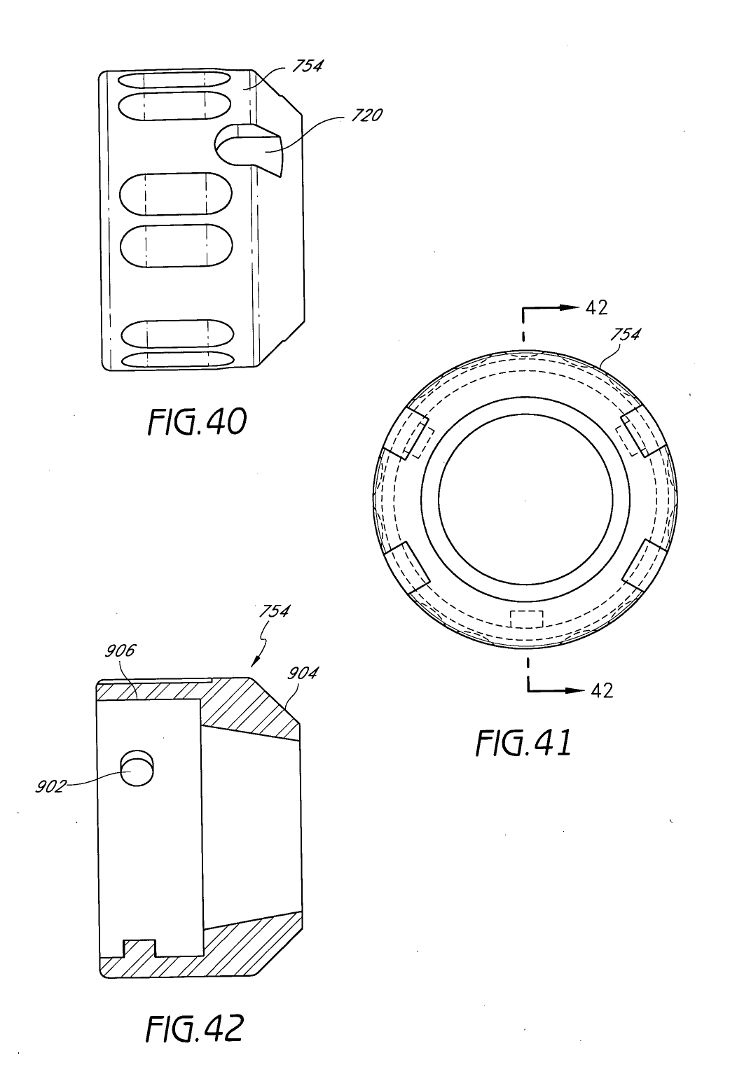

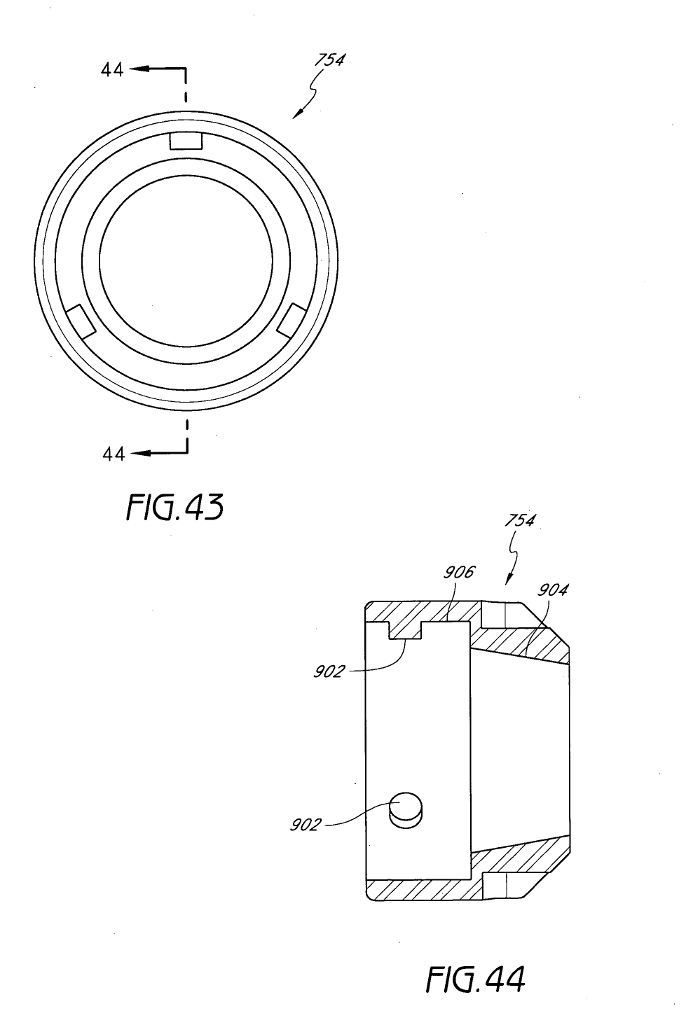

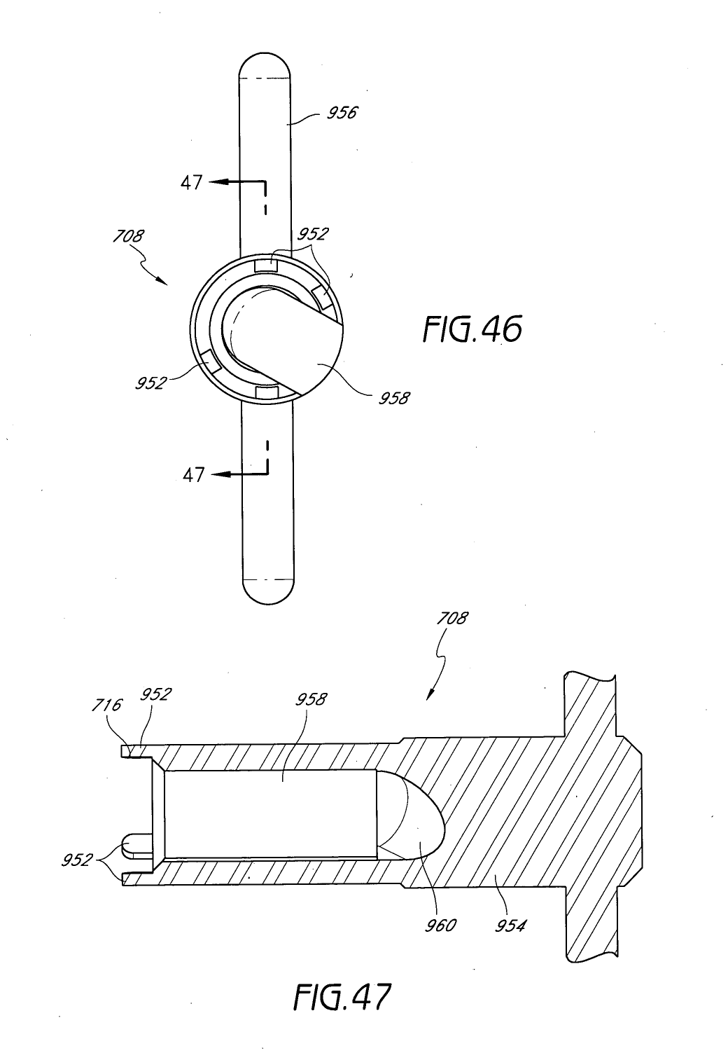

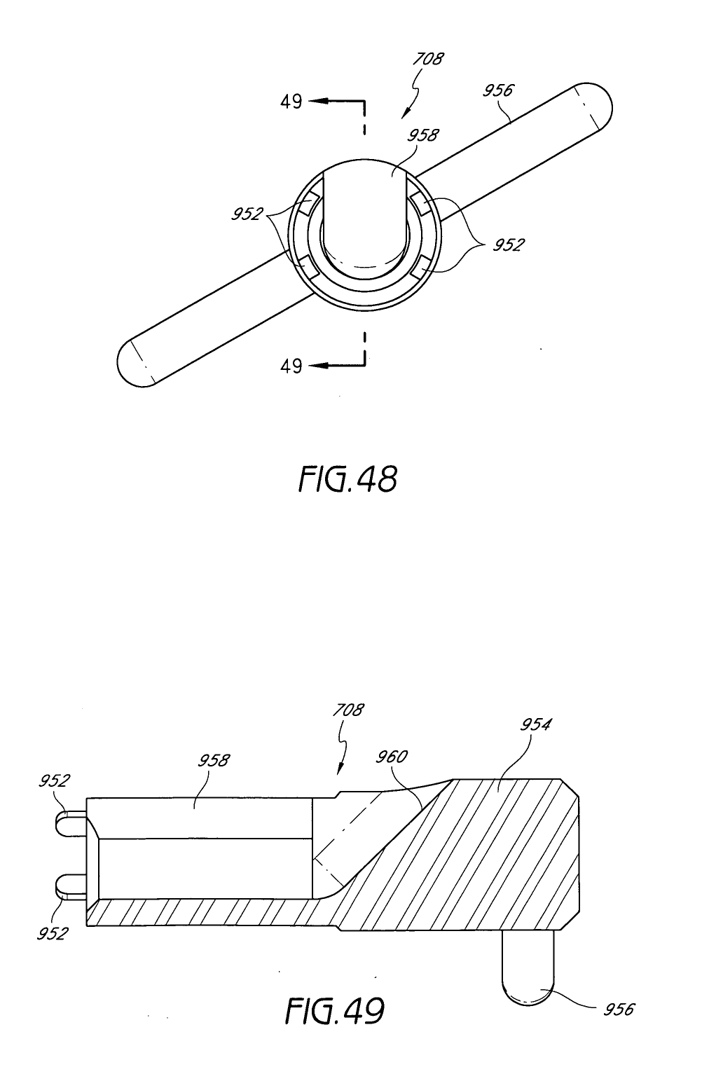

FIG. 1 shows that the outflow conduit 52 has a first end 66 that connects to the outlet 36 of the pump 32 and a second end 68 that connects with a second peripheral blood vessel. As discussed further below in connection with FIGS. 17-49, various systems, devices, and methods can be used to connect the first end 66 of the conduit 52 to the outlet 36 of the pump 32. These systems, devices, and methods are particularly useful in connection with heart assist and blood supplementation systems that are implantable. However, these systems, devices, and methods for connecting can advantageously couple any of the conduits, cannulae or catheters, or graft described herein or any similar conduits, cannulae or catheters, or graft with any other component, including the pumps disclosed herein and similar pumps. The outflow conduit 52 can be coupled with any suitable vessel, such as the left subclavian artery 24 of the patient 12, the right axillary artery, or any other peripheral or non-primary artery. In one application, the connection between the outflow conduit 52 and the second blood vessel is via an end-to-side anastomosis, although a side-to-side anastomosis connection might be used mid-stream of the conduit where the outflow conduit were connected at its second end to yet another blood vessel or at another location on the same blood vessel (neither shown). Preferably, the outflow conduit 52 is attached to the second blood vessel at an angle that results in the predominant flow of blood out of the pump 32 proximally toward the aorta 16 and the heart 14, such as is shown in FIG. 1, while still maintaining sufficient flow distally toward the hand to prevent limb ischemia.

In another embodiment, the inflow conduit 50 is connected to the first blood vessel via an end-to-side anastomosis, rather than via the inflow cannula 60. The inflow conduit 50 could also be coupled with the first blood vessel via a side-to-side anastomosis connection mid-stream of the conduit where the inflow conduit were connected at its second end to an additional blood vessel or at another location on the same blood vessel (neither shown). Further details of these arrangements and other related applications are described in U.S. application Ser. No. 10/289,467, filed Nov. 6, 2002, the entire contents of which is hereby incorporated by reference in its entirety and made a part of this specification.

In another embodiment, the outflow conduit 52 also is coupled with the second blood vessel via a cannula, as shown in FIG. 6. This connection may be achieved in a manner similar to that shown in FIG. 1 in connection with the first blood vessel.

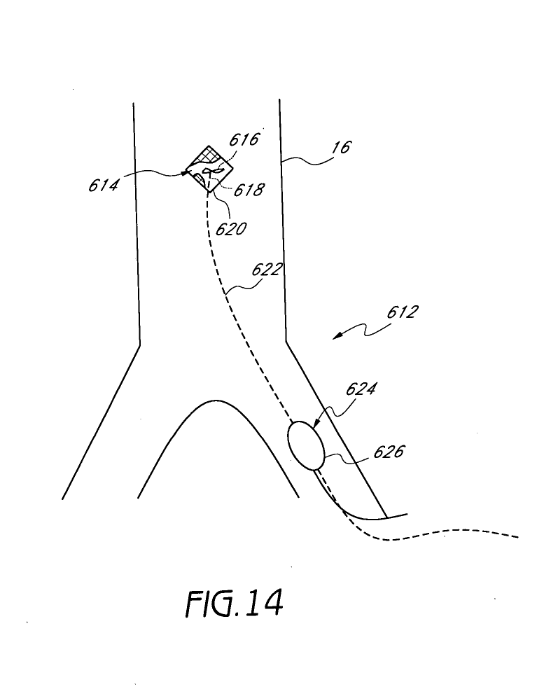

It is preferred that application of the heart assist system 10 to the peripheral or non-primary blood vessels be accomplished subcutaneously; e.g., at a shallow depth just below the skin or first muscle layer so as to avoid major invasive surgery. It is also preferred that the heart assist system 10 be applied extrathoracically to avoid the need to invade the patient's chest cavity. Where desired, the entire heart assist system 10 may be implanted within the patient 12, either extravascularly, e.g., as in FIG. 1, or at least partially intravascularly, e.g., as in FIGS. 14-16.

In the case of an extravascular application, the pump 32 may be implanted, for example, into the groin area, with the inflow conduit 50 fluidly connected subcutaneously to, for example, the femoral artery 26 proximate the pump 32. The outflow conduit would be tunneled subcutaneously through to, for example, the left subclavian artery 24. In an alternative arrangement, the pump 32 and associated drive and controller could be temporarily fastened to the exterior skin of the patient, with the inflow and outflow conduits 50, 52 connected percutaneously. In either case, the patient may be ambulatory without restriction of tethered lines.

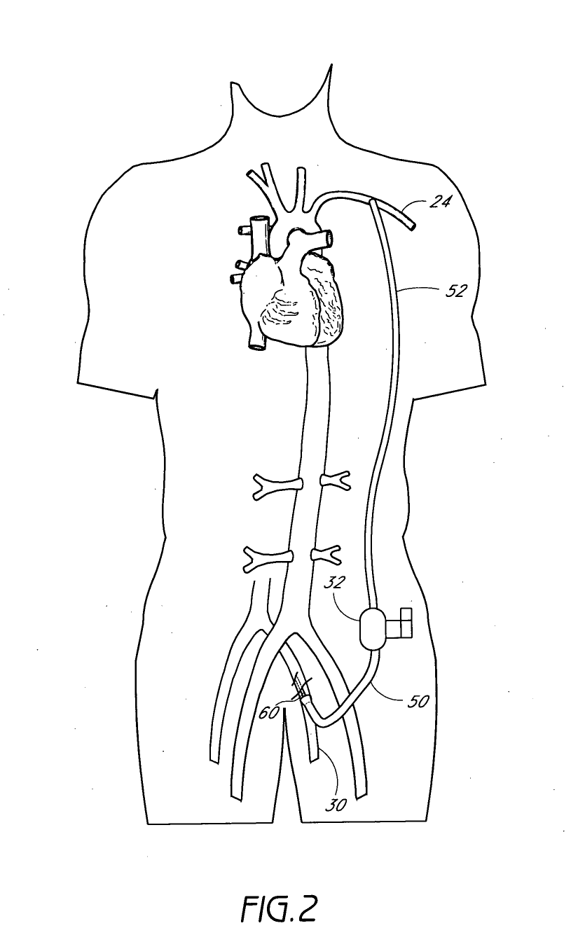

While the heart assist system 10 and other heart assist systems described herein may be applied to create an arterial-arterial flow path, given the nature of the heart assist systems, i. e., supplementation of circulation to meet organ demand, a venous-arterial flow path may also be used. For example, with reference to FIG. 2, one application of the heart assist system 10 couples the inflow conduit 50 with a non-primary vein of the patient 12, such as the left femoral vein 30. In this arrangement, the outflow conduit 50 may be fluidly coupled with one of the peripheral arteries, such as the left subclavian artery 24. Arterial-venous arrangements are contemplated as well. In those venous-arterial cases where the inflow is connected to a vein and the outflow is connected to an artery, the pump 32 should be sized to permit flow sufficiently small so that oxygen-deficient blood does not rise to unacceptable levels in the arteries. It should be appreciated that the connections to the non-primary veins could be by one or more approach described above for connecting to a non-primary artery. It should also be appreciated that the present invention could be applied as a venous-venous flow path, wherein the inflow and outflow are connected to separate peripheral veins. In addition, an alternative embodiment comprises two discrete pumps and conduit arrangements, one being applied as a venous-venous flow path, and the other as an arterial-arterial flow path.

When venous blood is mixed with arterial blood either at the inlet of the pump or the outlet of the pump the ratio of venous blood to arterial blood should be controlled to maintain an arterial saturation of a minimum of 80% at the pump inlet or outlet. Arterial saturation can be measured and/or monitored by pulse oximetry, laser doppler, colorimetry or other methods used to monitor blood oxygen saturation. The venous blood flow into the system can then be controlled by regulating the amount of blood allowed to pass through the conduit from the venous-side connection.

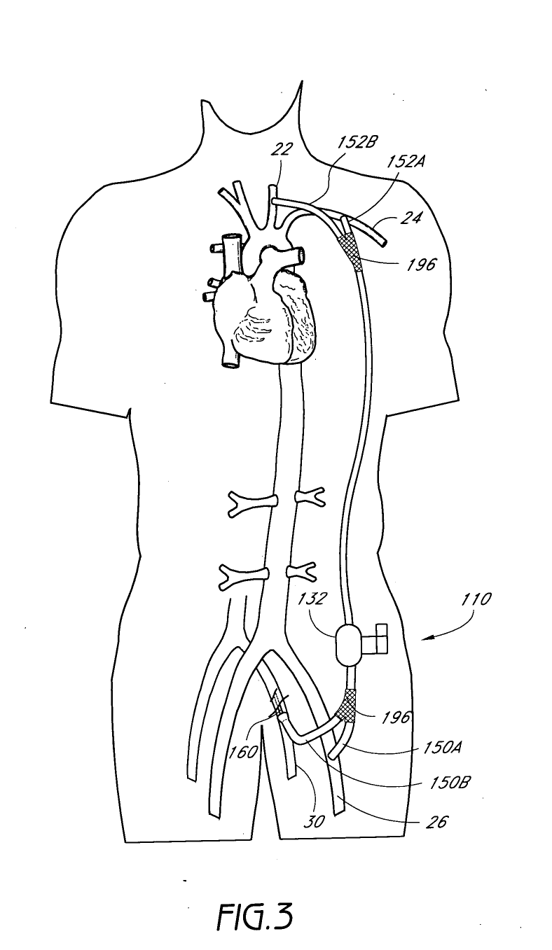

FIG. 3 shows another embodiment of a heart assist system 110 applied to the patient 12. For example, the heart assist system 110 includes a pump 132 in fluid communication with a plurality of inflow conduits 150A, 150B and a plurality of outflow conduits 152A, 152B. Each pair of conduits converges at a generally Y-shaped convergence 196 that converges the flow at the inflow end and diverges the flow at the outflow end. Each conduit may be connected to a separate peripheral blood vessel, although it is possible to have two connections to the same blood vessel at remote locations. In one arrangement, all four conduits are connected to peripheral arteries. In another arrangement, one or more of the conduits could be connected to veins. In the arrangement of FIG. 3, the inflow conduit 150A is connected to the left femoral artery 26 while the inflow conduit 150B is connected to the left femoral vein 30. The outflow conduit 152A is connected to the left subclavian artery 24 while the outflow conduit 152B is connected to the left carotid artery 22. Preferably at least one of the conduits 150A, 150B, 152A, and 152B is coupled with a corresponding vessel via a cannula. In the illustrated embodiment, the inflow conduit 150B is coupled with the left femoral vein 30 via a cannula 160. The cannula 160 is coupled in a manner similar to that shown in FIG. 2 and described in connection with the cannula 60.

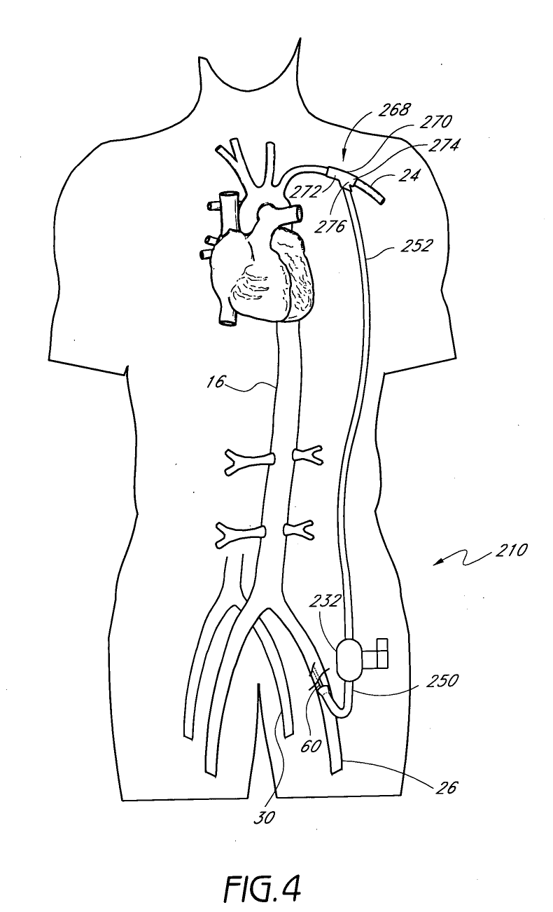

The connections of any or all of the conduits of the system 110 to the blood vessels may be via an anastomosis connection or via a connector, as described below in connection with FIG. 4. In addition, the embodiment of FIG. 3 may be applied to any combination of peripheral blood vessels that would best suit the patient's condition. For example, it may be desired to have one inflow conduit and two outflow conduits or vice versa. It should be noted that more than two conduits may be used on the inflow or outflow side, where the number of inflow conduits is not necessarily equal to the number of outflow conduits.

It is contemplated that, where an anastomosis connection is not desired, a connector may be used to connect at least one of the inflow conduit and the outflow conduit to a peripheral blood vessel. With reference to FIG. 4, an embodiment of a heart assist system 210 is shown, wherein an outflow conduit 252 is connected to a non-primary blood vessel, e.g., the left subclavian artery 24, via a connector 268 that comprises a three-opening fitting. In one embodiment, the connector 268 comprises an intra-vascular, generally T-shaped fitting 270 having a proximal end 272 (relative to the flow of blood in the left axillary artery and therethrough), a distal end 274, and an angled divergence 276 permitting connection to the outflow conduit 252 and the left subclavian artery 24. The proximal and distal ends 274, 276 of the fittings 272 permit connection to the blood vessel into which the fitting is positioned, e.g., the left subclavian artery 24. The angle of divergence 276 of the fittings 272 may be 90 degrees or less in either direction from the axis of flow through the blood vessel, as optimally selected to generate the needed flow distally toward the hand to prevent limb ischemia, and to insure sufficient flow and pressure toward the aorta to provide the circulatory assistance and workload reduction needed while minimizing or avoiding endothelial damage to the blood vessel. In another embodiment, the connector 268 is a sleeve (not shown) that surrounds and attaches to the outside of the non-primary blood vessel where, within the interior of the sleeve, a port to the blood vessel is provided to permit blood flow from the outflow conduit 252 when the conduit 252 is connected to the connector 268.

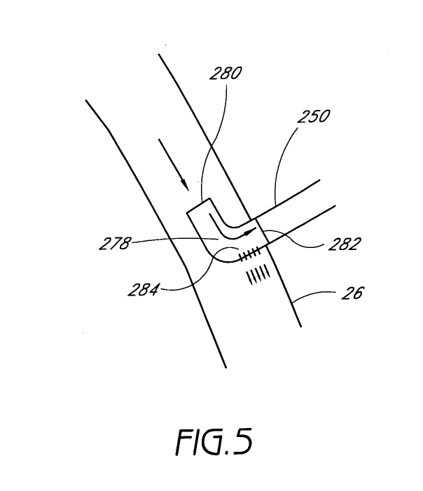

Other types of connectors having other configurations are contemplated that may avoid the need for an anastomosis connection or that permit connection of the conduit(s) to the blood vessel(s). For example, it is contemplated that an L-shaped connector be used if it is desired to withdraw blood more predominantly from one direction of a peripheral vessel or to direct blood more predominantly into a peripheral vessel. Referring to FIG. 5, the inflow conduit 250 is fluidly connected to a peripheral vessel, for example, the left femoral artery 26, using an L-shaped connector 278. Of course the system 210 could be configured so that the outflow conduit 252 is coupled to a non-primary vessel via the L-shaped connector 278 and the inflow conduit 250 is coupled via a cannula, as shown in FIG. 3. The L-shaped connector 278 has an inlet port 280 at a proximal end and an outlet port 282 through which blood flows into the inflow conduit 250. The L-shaped connector 278 also has an arrangement of holes 284 within a wall positioned at a distal end opposite the inlet port 280 so that some of the flow drawn into the L-shaped connector 278 is diverted through the holes 284, particularly downstream of the L-shaped connector 278, as in this application. A single hole 284 in the wall could also be effective, depending upon size and placement. The L-shaped connector 278 may be a deformable L-shaped catheter percutaneously applied to the blood vessel or, in an alternative embodiment, be connected directly to the walls of the blood vessel for more long term application. By directing some blood flow downstream of the L-shaped connector 278 during withdrawal of blood from the vessel, ischemic damage downstream from the connector may be avoided. Such ischemic damage might otherwise occur if the majority of the blood flowing into the L-shaped connector 278 were diverted from the blood vessel into the inflow conduit 252. It is also contemplated that a connection to the blood vessels might be made via a cannula, wherein the cannula is implanted, along with the inflow and outflow conduits.

One advantage of discrete connectors manifests in their application to patients with chronic CHF. A connector eliminates a need for an anastomosis connection between the conduits 250, 252 and the peripheral blood vessels where it is desired to remove and/or replace the system more than one time. The connectors could be applied to the first and second blood vessels semi-permanently, with an end cap applied to the divergence for later quick-connection of the present invention system to the patient. In this regard, a patient might experience the benefit of the heart assist systems described herein periodically, without having to reconnect and redisconnect the conduits 250, 252 from the blood vessels via an anastomosis procedure each time. Each time it is desired to implement any of the embodiments of the heart assist system, the end caps would be removed and a conduit attached to the connector(s) quickly.

In the preferred embodiment of the connector 268, the divergence 276 is oriented at an acute angle significantly less than 90 degrees from the axis of the T-shaped fitting 270, as shown in FIG. 4, so that a majority of the blood flowing through the outflow conduit 252 into the blood vessel (e.g., left subclavian artery 24) flows in a direction proximally toward the heart 14, rather than in the distal direction. In an alternative embodiment, the proximal end 272 of the T-shaped fitting 270 may have a diameter larger than the diameter of the distal end 274, without need of having an angled divergence, to achieve the same result.

With or without a connector, with blood flow directed proximally toward the aorta 16, the result may be concurrent flow down the descending aorta, which will result in the reduction of afterload, impedence, and/or reducing left ventricular end diastolic pressure and volume (preload). Thus, the heart assist systems described herein may be applied so to reduce the afterload on the patient's heart, permitting at least partial if not complete CHF recovery, while supplementing blood circulation. Concurrent flow depends upon the phase of operation of the pulsatile pump and the choice of second blood vessel to which the outflow conduit is connected.

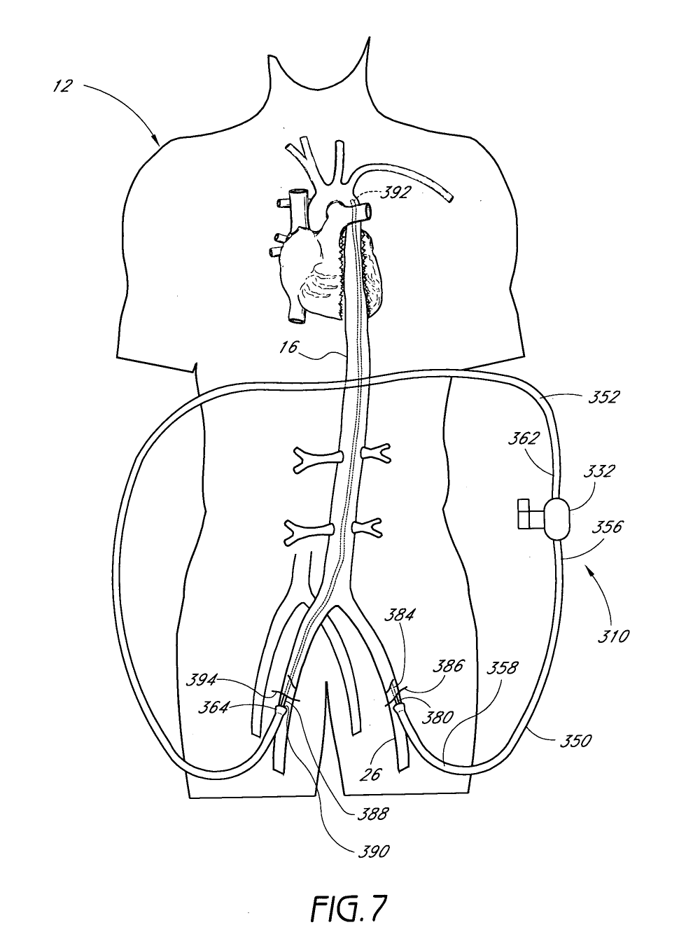

A partial external application of the heart assist systems is contemplated where a patient with heart failure is suffering an acute decompensation episode; i.e., is not expected to last long, or in the earlier stages of heart failure (where the patient is in New York Heart Association Classification (NYHAC) functional classes II or III). With reference to FIGS. 6 and 7, another embodiment of a heart assist system 310 is applied percutaneously to a patient 312 to connect two non-primary blood vessels wherein a pump 332 and its associated driving means and controls are employed extracorporeally. The pump 332 has an inflow conduit 350 and an outflow conduit 352 associated therewith for connection to two non-primary blood vessels. The inflow conduit 350 has a first end 356 and a second end 358 wherein the second end 358 is connected to a first non-primary blood vessel (e.g., femoral artery 26) by way of an inflow cannula 380. The inflow cannula 380 has a first end 382 sealably connected to the second end 358 of the inflow conduit 350. The inflow cannula 380 also has a second end 384 that is inserted through a surgical opening 386 or an introducer sheath (not shown) and into the blood vessel (e.g., the left femoral artery 26).

Similarly, the outflow conduit 352 has a first end 362 and a second end 364 wherein the second end 364 is connected to a second non-primary blood vessel (e.g., the left subclavian artery 24, as shown in FIG. 6, or the right femoral artery 28, as shown in FIG. 7) by way of an outflow cannula 388. Like the inflow cannula 380, the outflow cannula 388 has a first end 390 sealably connected to the second end 364 of the outflow conduit 352. The outflow cannula 388 also has a second end 392 that is inserted through surgical opening 394 or an introducer sheath (not shown) and into the second blood vessel (e.g., the left subclavian artery 24 or the right femoral artery 28).

As shown in FIG. 7, the second end 392 of the outflow cannula 388 may extend well into the aorta 16 of the patient 12, for example, proximal to the left subclavian artery. If desired, it may also terminate within the left subclavian artery or the left axillary artery, or in other blood vessels, such as the mesenteric or renal arteries (not shown), where in either case, the outflow cannula 388 has passed through at least a portion of a primary artery (in this case, the aorta 16). Also, if desired, blood drawn into the extracardiac system 310 described herein may originate from the descending aorta (or an artery branching therefrom) and be directed into a blood vessel that is neither the aorta nor pulmonary artery. By use of a percutaneous application, the heart assist system 310 may be applied temporarily without the need to implant any aspect thereof or to make anastomosis connections to the blood vessels.

An alternative variation of the embodiment of FIG. 6 may be used where it is desired to treat a patient periodically, but for short periods of time each occasion and without the use of special connectors. With this variation, it is contemplated that the second ends of the inflow and outflow conduits 350, 352 be more permanently connected to the associated blood vessels via, for example, an anastomosis connection, wherein a portion of each conduit proximate to the blood vessel connection is implanted percutaneously with a removable cap enclosing the externally-exposed first end (or an intervening end thereof) of the conduit external to the patient. When it is desired to provide a circulatory flow path to supplement blood flow, the removable cap on each exposed percutaneously-positioned conduit could be removed and the pump (or the pump with a length of inflow and/or outflow conduit attached thereto) inserted between the exposed percutaneous conduits. In this regard, a patient may experience the benefit of the present invention periodically, without having to reconnect and redisconnect the conduits from the blood vessels each time.

Specific methods of applying this alternative embodiment may further comprise coupling the inflow conduit 352 upstream of the outflow conduit 350 (as shown in FIG. 8), although the reverse arrangement is also contemplated. It is also contemplated that either the cannula 380 coupled with the inflow conduit 350 or the cannula 388 coupled with the outflow conduit 352 may extend through the non-primary blood vessel to a second blood vessel (e.g., through the left femoral artery 26 to the aorta 16 proximate the renal branch) so that blood may be directed from the non-primary blood vessel to the second blood or vice versa.

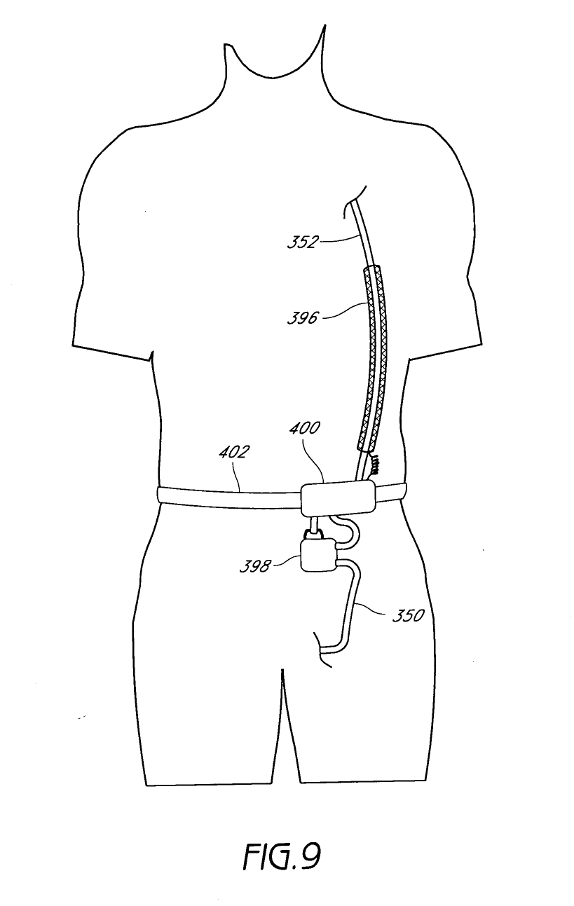

It is contemplated that a means for minimizing the loss of thermal energy in the patient's blood be provided where any of the heart assist systems described herein are applied extracorporeally. Such means for minimizing the loss of thermal energy may comprise, for example, a heated bath through which the inflow and outflow conduits pass or, alternatively, thermal elements secured to the exterior of the inflow and outflow conduits. Referring to FIG. 9, one embodiment comprises an insulating wrap 396 surrounding the outflow conduit 352 having one or more thermal elements passing therethrough. The elements may be powered, for example, by a battery (not shown). One advantage of thermal elements is that the patient may be ambulatory, if desired. Other means that are known by persons of ordinary skill in the art for ensuring that the temperature of the patient's blood remains at acceptable levels while travelling extracorporeally are also contemplated.

If desired, the present inventive system may further comprise a reservoir that is either contained within or in fluid communication with the inflow conduit. This reservoir is preferably made of materials that are nonthrombogenic. Referring to FIG. 9, a reservoir 398 is positioned fluidly in line with the inflow conduit 350. The reservoir 398 serves to sustain adequate blood in the system when the pump demand exceeds momentarily the volume of blood available in the peripheral blood vessel in which the inflow conduit resides until the pump output can be adjusted. The reservoir 398 reduces the risk of excessive drainage of blood from the peripheral blood vessel, which may occur when cardiac output falls farther than the already diminished baseline level of cardiac output, or when there is systemic vasodilation, as can occur, for example, with septic shock. It is contemplated that the reservoir 398 would be primed with an acceptable solution, such as saline, when the present system is first applied to the patient.

As explained above, one of the advantages of several embodiments of the heart assist system is that such systems permit the patient to be ambulatory. If desired, the systems may be designed portably so that it may be carried directly on the patient. Referring to FIG. 9, this may be accomplished through the use of a portable case 400 with a belt strap 402 to house the pump, power supply and/or the controller, along with certain portions of the inflow and/or outflow conduits, if necessary. It may also be accomplished with a shoulder strap or other techniques, such as a backpack or a fanny pack, that permit effective portability. As shown in FIG. 9, blood is drawn through the inflow conduit 350 into a pump contained within the portable case 400, where it is discharged into the outflow conduit 352 back into the patient.

B. Heart Assist Systems and Methods Employing Single-site Application

As discussed above, heart assist systems can be applied to a patient through a single cannulation site. Such single-site systems can be configured with a pump located outside the vasculature of a patient, e.g., as extravascular pumping systems, inside the vasculature of the patient, e.g., as intravascular systems, or a hybrid thereof, e.g., partially inside and partially outside the vasculature of the patient.

1. Single-Site Application of Extravascular Pumping Systems

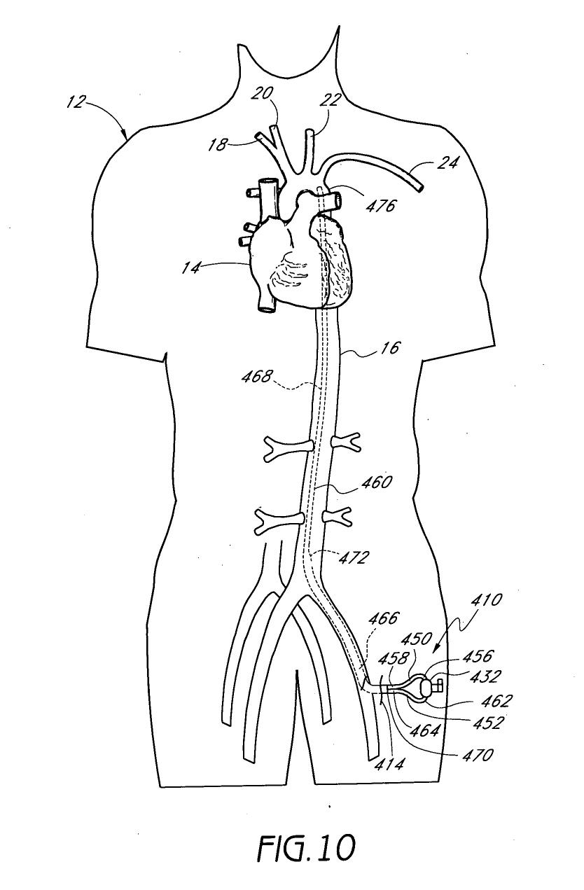

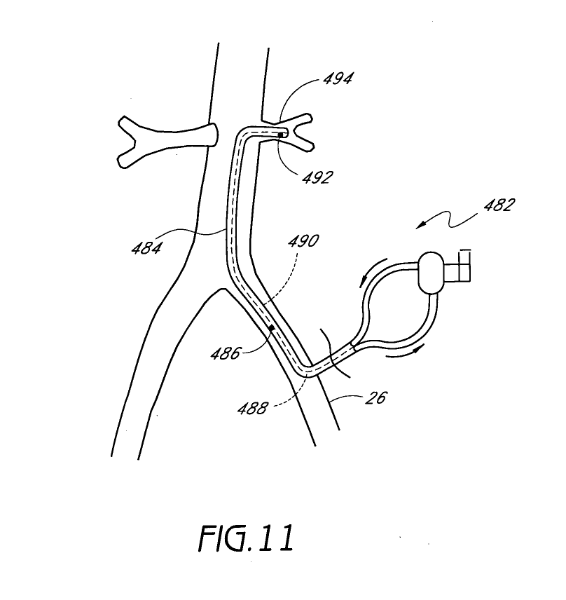

FIGS. 10 and 11 illustrate extracardiac heart assist systems that employ an extravascular pump and that can be applied through as a single-site system. FIG. 10 shows a system 410 that is applied to a patient 12 through a single cannulation site 414 while inflow and outflow conduits fluidly communicate with non-primary vessels. The heart assist system 410 is applied to the patient 12 percutaneously through a single-site to couple two blood vessels with a pump 432. The pump 432 can have any of the features described in connection the pump 32. The pump 432 has an inflow conduit 450 and an outflow conduit 452 associated therewith. The inflow conduit 450 has a first end 456 and a second end 458. The first end 456 of the inflow conduit 450 is connected to the inlet of the pump 432 and the second end 458 of the inflow conduit 450 is fluidly coupled with a first non-primary blood vessel (e.g., the femoral artery 26) by way of a multilumen cannula 460. Similarly, the outflow conduit 452 has a first end 462 and a second end 464. The first end 462 of the outflow conduit 452 is connected to the outlet of the pump 432 and the second end 464 of the outflow conduit 452 is fluidly coupled with a second blood vessel (e.g., the descending aorta 16) by way of the multilumen cannula 460.

In one embodiment, the multilumen cannula 460 includes a first lumen 466 and a second lumen 468. The first lumen 466 extends from a proximal end 470 of the multilumen cannula 460 to a first distal end 472. The second lumen 468 extends from the proximal end 470 to a second distal end 474. In the illustrated embodiment, the second end 458 of the inflow conduit 450 is connected to the first lumen 466 of the multilumen cannula 460 and the second end 464 of the outflow conduit 452 is connected to the second lumen 468 of the multilumen cannula 460.

Where there is a desire for the patient 12 to be ambulatory, the multilumen cannula 460 preferably is made of material sufficiently flexible and resilient to permit the patient 12 to be comfortably move about while the multilumen cannula 460 is indwelling in the patient's blood vessels without causing any vascular trauma.

The application shown in FIG. 10 and described above results in flow from the first distal end 472 to the second distal end 474. Of course, the flow direction may be reversed using the same arrangement, resulting in flow from the distal end 474 to the distal end 472. In some applications, the system 410 is applied in an arterial-arterial fashion. For example, as illustrated, the multilumen cannula 460 can be inserted into the left femoral artery 26 of the patient 12 and guided superiorly through the descending aorta to one of numerous locations. In one application, the multilumen cannula 460 can be advanced until the distal end 474 is located in the aortic arch 476 of the patient 12. The blood could discharge, for example, directly into the descending aorta proximate an arterial branch, such as the left subclavian artery or directly into the peripheral mesenteric artery (not shown).

The pump 432 draws blood from the patient's vascular system in the area near the distal end 472 and into the lumen 466. This blood is further drawn into the lumen of the conduit 450 and into the pump 432. The pump 432 then expels the blood into the lumen of the outflow conduit 452, which carries the blood into the lumen 468 of the multilumen cannula 460 and back into the patient's vascular system in the area near the distal end 474.

FIG. 11 shows another embodiment of a heart assist system 482 that is similar to the heart assist system 410, except as set forth below. The system 482 employs a multilumen cannula 484. In one application, the multilumen cannula 484 is inserted into the left femoral artery 26 and guided superiorly through the descending aorta to one of numerous locations. Preferably, the multilumen cannula 484 has an inflow port 486 that is positioned in one application within the left femoral artery 26 when the cannula 484 is fully inserted so that blood drawn from the left femoral artery 26 is directed through the inflow port 486 into a first lumen 488 in the cannula 484. The inflow port 486 can also be positioned in any other suitable location within the vasculature, described herein or apparent to one skilled in the art. This blood is then pumped through a second lumen 490 in the cannula 484 and out through an outflow port 492 at the distal end of the cannula 484. The outflow port 492 may be situated within, for example, a mesenteric artery 494 such that blood flow results from the left femoral artery 26 to the mesenteric artery 494. The blood could discharge, for example, directly into the descending aorta proximate an arterial branch, such as the renal arteries, the left subclavian artery, or directly into the peripheral mesenteric artery 494, as illustrated in FIG. 11. Where there is a desire for the patient to be ambulatory, the multilumen cannula 484 preferably is made of material sufficiently flexible and resilient to permit the patient 12 to comfortably move about while the cannula 484 is indwelling in the patient's blood vessels without causing any vascular trauma.

Further details of the multilumen cannula 460 are described below in connection with FIG. 11. Additional details also may be found in U.S. patent application Ser. No. 10/078,283, filed Feb. 14, 2002, entitled A MULTILUMEN CATHETER FOR MINIMIZING LIMB ISCHEMIA and in U.S. patent application Ser. No. 10/706,346, filed Nov. 12, 2003, entitled CANNULAE HAVING REDIRECTING TIP, which are hereby expressly incorporated by reference in its entirety and made a part of this specification.

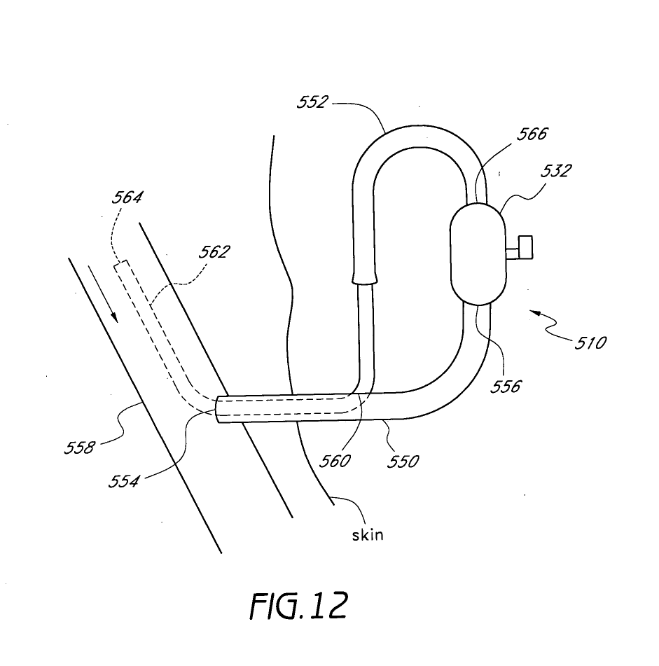

FIG. 12 shows another heart assist system 510 that takes further advantage of the supplemental blood perfusion and heart load reduction benefits while remaining minimally invasive in application. The heart assist system 510 is an extracardiac pumping system that includes a pump 532, an inflow conduit 550 and an outflow conduit 552. In the illustrated embodiment, the inflow conduit 550 comprises a vascular graft. The vascular graft conduit 550 and the outflow conduit 552 are fluidly coupled to pump 532. The pump 532 is configured to pump blood through the patient at subcardiac volumetric rates, and has an average flow rate that, during normal operation thereof, is substantially below that of the patient's heart when healthy. In one variation, the pump 532 may be a rotary pump. Other pumps described herein, or any other suitable pump can also be used in the extracardiac pumping system 510. In one application, the pump 532 is configured so as to be implantable.

The vascular graft 550 has a first end 554 and a second end 556. The first end 554 is sized and configured to couple to a non-primary blood vessel 558 subcutaneously to permit application of the extracardiac pumping system 510 in a minimally-invasive procedure. In one application, the vascular graft conduit 550 is configured to couple to the blood vessel 558 via an anastomosis connection. The second end 556 of the vascular graft 550 is fluidly coupled to the pump 532 to conduct blood between the non-primary blood vessel 558 and the pump 532. In the embodiment shown, the second end 556 is directly connected to the pump 532, but, as discussed above in connection with other embodiments, intervening fluid conducting elements may be interposed between the second end 556 of the vascular graft 550 and the pump 532. Examples of arrangements of vascular graft conduits may be found in U.S. application Ser. No. 09/780,083, filed Feb. 9, 2001, entitled EXTRA-CORPOREAL VASCULAR CONDUIT, which is hereby incorporated by reference in its entirety and made a part of this specification.

FIG. 12 illustrates that the present inventive embodiment further comprises means for coupling the outflow conduit 552 to the vascular graft 550, which may comprise in one embodiment an insertion site 560. In the illustrated embodiment, the insertion site 560 is located between the first end 554 and the second end 556 of the vascular graft 550. The outflow conduit 552 preferably is coupled with a cannula 562.

The insertion site 560 is configured to receive the cannula 562 therethrough in a sealable manner in the illustrated embodiment. In another embodiment, the insertion site 560 is configured to receive the outflow conduit 552 directly. The cannula 562 includes a first end 564 sized and configured to be inserted through the insertion site 560, through the cannula 550, and through the non-primary blood vessel 558. The conduit 552 has a second end 566 fluidly coupled to the pump 532 to conduct blood between the pump 532 and the blood vessel 558.

The extracardiac pumping system 510 can be applied to a patient, as shown in FIG. 12, so that the outflow conduit 552 provides fluid communication between the pump 532 and a location upstream or downstream of the location where the cannula 562 enters the non-primary blood vessel 558. In another application, the cannula 562 is directed through the blood vessel to a different blood vessel, upstream or downstream thereof. Although the vascular graft 550 is described above as an “inflow conduit” and the conduit 552 is described above as an “outflow conduit,” in another application of this embodiment, the blood flow through the pumping system 510 is reversed (i.e., the pump 532 pumps blood in the opposite direction), whereby the vascular graft 550 is an outflow conduit and the conduit 552 is an inflow conduit.

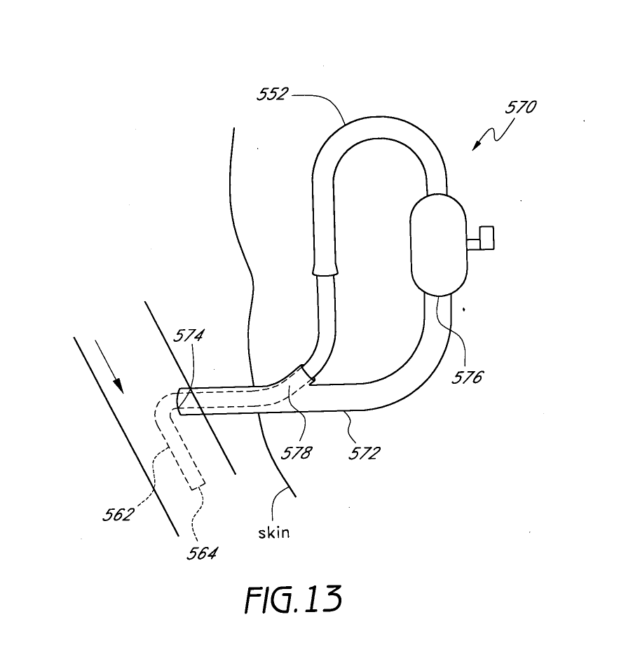

FIG. 13 shows a variation of the extracardiac pumping system shown in FIG. 12. In particular, a heart assist system 570 includes an inflow conduit 572 that comprises a first end 574, a second end 576, and means for connecting the outflow conduit 552 to the inflow conduit 572. In one embodiment, the inflow conduit 572 comprises a vascular graft. The extracardiac pumping system 570 is otherwise similar to the extracardiac pumping system 510. The means for connecting the conduit 552 to the inflow conduit 572 may comprise a branched portion 578. In one embodiment, the branched portion 578 is located between the first end 574 and the second end 576. The branched portion 578 is configured to sealably receive the distal end 564 of the outflow conduit 552. Where, as shown, the first end 564 of the outflow conduit 552 comprises the cannula 562, the branched portion 578 is configured to receive the cannula 562. The inflow conduit 572 of this arrangement comprises in part a multilumen cannula, where the internal lumen extends into the blood vessel 558. Other multilumen catheter arrangements are shown in U.S. application Ser. No. 10/078,283, incorporated by reference herein above.

2. Single-Site Application of Intravascular Pumping Systems

FIGS. 14-16 illustrate extracardiac heart assist systems that employ intravascular pumping systems. Such systems take further advantage of the supplemental blood perfusion and heart load reduction benefits discussed above while remaining minimally invasive in application. Specifically, it is contemplated to provide an extracardiac pumping system that comprises a pump that is sized and configured to be at least partially implanted intravascularly in any location desirable to achieve those benefits, while being insertable through a non-primary vessel.

FIG. 14 shows a heart assist system 612 that includes a pumping means 614 comprising preferably one or more rotatable impeller blades 616, although other types of pumping means 614 are contemplated, such as an archimedes screw, a worm pump, or other means by which blood may be directed axially along the pumping means from a location upstream of an inlet to the pumping means to a location downstream of an outlet from the pumping means. Where one or more impeller blades 616 are used, such as in a rotary pump, such impeller blades 616 may be supported helically or otherwise on a shaft 618 within a housing 620. The housing 620 may be open, as shown, in which the walls of the housing 620 are open to blood flow therethrough. The housing 620 may be entirely closed, if desired, except for an inlet and outlet (not shown) to permit blood flow therethrough in a more channel fashion. The heart assist system 612 serves to supplement the kinetic energy of the blood flow through the blood vessel in which the pump is positioned, e.g., the aorta 16.

The impeller blade(s) 616 of the pumping means 614 of this embodiment may be driven in one or a number of ways known to persons of ordinary skill in the art. In the embodiment shown in FIG. 14, the impeller blade(s) 616 are driven mechanically via a rotatable cable or drive wire 622 by driving means 624, the latter of which may be positioned corporeally (intra- or extra-vascularly) or extracorporeally. As shown, the driving means 624 may comprise a motor 626 to which energy is supplied directly via an associated battery or an external power source, in a manner described in more detail herein. It is also contemplated that the impeller blade(s) 616 be driven electromagnetically through an internal or external electromagnetic drive. Preferably, a controller (not shown) is provided in association with this embodiment so that the pumping means 614 may be controlled to operate in a continuous and/or pulsatile fashion, as described herein.

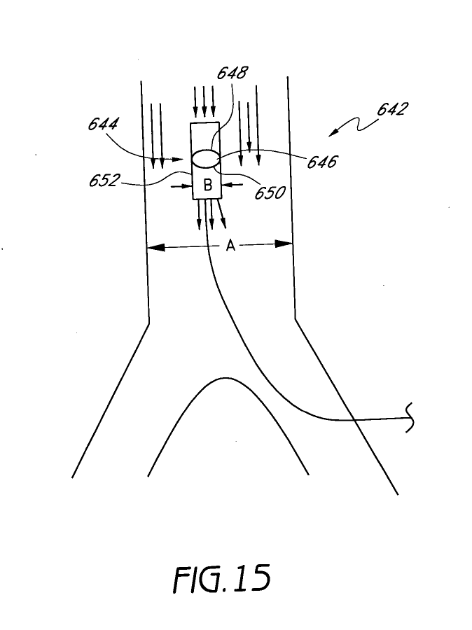

Variations of the intravascular embodiment of FIG. 14 are shown in FIGS. 15 and 16. In the embodiment of FIG. 15, an intrasvascular extracardiac system 642 comprising a pumping means 644, which may be one of several means described herein. The pumping means 644 may be driven in any suitable manner, including means sized and configured to be implantable and, if desired, implantable intravascularly, e.g., as discussed above. For a blood vessel (e.g., descending aorta) having a diameter “A”, the pumping means 644 preferably has a meaningfully smaller diameter “B”. The pumping means 644 may comprise a pump 646 having an inlet 648 and an outlet 650. The pumping means 644 also comprises a pump driven mechanically by a suitable drive arrangement in one embodiment. Although the vertical arrows in FIG. 15 illustrate that the pumping means 644 pumps blood in the same direction as the flow of blood in the vessel, the pumping means 644 could be reversed to pump blood in a direction generally opposite of the flow in the vessel.

In one embodiment, the pumping means 644 also includes a conduit 652 in which the pump 646 is housed. The conduit 652 may be relatively short, as shown, or may extend well within the designated blood vessel or even into an adjoining or remote blood vessel at either the inlet end, the outlet end, or both. The intravascular extracardiac system 642 may further comprise an additional parallel-flow conduit, as discussed below in connection with the system of FIG. 16.

The intrasvascular extracardiac system 642 may further comprise inflow and/or outflow conduits or cannulae (not shown) fluidly connected to the pumping means 644, e.g., to the inlet and outlet of pump 646. Any suitable conduit or cannula can be employed.

In another embodiment, an intrasvascular pumping means 644 may be positioned within one lumen of a multilumen catheter so that, for example, where the catheter is applied at the left femoral artery, a first lumen may extend into the aorta proximate the left subclavian and the pumping means may reside at any point within the first lumen, and the second lumen may extend much shorter just into the left femoral or left iliac. Such a system is described in greater detail in U.S. application Ser. No. 10/078,283, incorporated by reference herein above.

FIG. 16 shows a variation of the heart assist system of FIG. 15. In particular the intravascular system may further comprise an additional conduit 660 positioned preferably proximate the pumping means 644 to provide a defined flow path for blood flow axially parallel to the blood flowing through the pumping means 644. In the case of the pumping means 644 of FIG. 16, the means comprises a rotatable cable 662 having blood directing means 664 supported therein for directing blood axially along the cable. Other types of pumping means are also contemplated, if desired, for use with the additional conduit 660.

Further details of intravascular pumping systems may be found in U.S. patent application Ser. No. 10/686,040, filed Oct. 15, 2003, which is hereby incorporated by reference herein in its entirety.

C. Potential Enhancement of Systemic Arterial Blood Mixing

One of the advantages of the present invention is its potential to enhance mixing of systemic arterial blood, particularly in the aorta. Such enhanced mixing ensures the delivery of blood with higher oxygen-carrying capacity to organs supplied by arterial side branches off of the aorta. A method of enhancing mixing utilizing the present invention preferably includes taking steps to assess certain parameters of the patient and then to determine the minimum output of the pump that, when combined with the heart output, ensures turbulent flow in the aorta, thereby enhancing blood mixing.

Blood flow in the aortic arch during normal cardiac output may be characterized as turbulent in the end systolic phase. It is known that turbulence in a flow of fluid through pipes and vessels enhances the uniform distribution of particles within the fluid. It is believed that turbulence in the descending aorta enhances the homogeneity of blood cell distribution in the aorta. It is also known that laminar flow of viscous fluids leads to a higher concentration of particulate in the central portion of pipes and vessels through which the fluid flows. It is believed that, in low flow states such as that experienced during heart failure, there is reduced or inadequate mixing of blood cells leading to a lower concentration of nutrients at the branches of the aorta to peripheral organs and tissues. As a result, the blood flowing into branch arteries off of the aorta will likely have a lower hematocrit, especially that flowing into the renal arteries, the celiac trunk, the spinal arteries, and the superior and inferior mesenteric arteries. That is because these branches draw from the periphery of the aorta The net effect of this phenomenon is that the blood flowing into these branch arteries has a lower oxygen-carrying capacity, because oxygen-carrying capacity is directly proportional to both hematocrit and the fractional O₂ saturation of hemoglobin. Under those circumstances, it is very possible that these organs will experience ischemia-related pathology.

The phenomenon of blood streaming in the aorta, and the resultant inadequate mixing of blood resulting in central lumenal concentration of blood cells, is believed to occur when the Reynolds number (N_{R}) for the blood flow in the aorta is below 2300. To help ensure that adequate mixing of blood will occur in the aorta to prevent blood cells from concentrating in the center of the lumen, a method of applying the present invention to a patient may also include steps to adjust the output of the pump to attain turbulent flow within the descending aorta upstream of the organ branches; i.e., flow exhibiting a peak Reynolds number of at least 2300 within a complete cycle of systole and diastole. Because flow through a patient is pulsatile in nature, and not continuous, consideration must be given to how frequently the blood flow through the aorta has reached a certain desired velocity and, thus, a desired Reynolds number. The method contemplated herein, therefore, should also include the step of calculating the average Womersley number (N_{W}), which is a function of the frequency of the patient's heart beat. It is desired that a peak Reynolds number of at least 2300 is attained when the corresponding Womersley number for the same blood flow is approximately 6 or above.

More specifically, the method may comprise calculating the Reynolds number for the blood flow in the descending aorta by determining the blood vessel diameter and both the velocity and viscosity of the fluid flowing through the aorta. The Reynolds number may be calculated pursuant to the following equation:

$${N}_{R} = \frac{V \cdot d}{υ}$$

where: V=the velocity of the fluid; d=the diameter of the vessel; and υ=the viscosity of the fluid. The velocity of the blood flowing through the aorta is a function of the cross-sectional area of the aorta and the volume of flow therethrough, the latter of which is contributed both by the patient's own cardiac output and by the output of the pump of the present invention. Velocity may be calculated by the following equation:

$$V = \frac{Q}{π {r}^{2}}$$

where Q=the volume of blood flowing through the blood vessel per unit time, e.g., the aorta, and r=radius of the aorta. If the relationship between the pump output and the velocity is already known or independently determinable, the volume of blood flow Q may consist only of the patient's cardiac output, with the knowledge that that output will be supplemented by the subcardiac pump that is part of the present invention. If desired, however, the present system can be implemented and applied to the patient first, before calculating Q, which would consist of the combination of cardiac output and the pump output.

The Womersley number may be calculated as follows:
N_{W}=r\sqrt{2πω/υ}

where r is the radius of the vessel being assessed, ω is the frequency of the patient's heartbeat, and υ=the viscosity of the fluid. For a peak Reynolds number of at least 2300, a Womersley number of at least 6 is preferred, although a value as low as 5 would be acceptable.

By determining (i) the viscosity of the patient's blood, which is normally about 3.0 mm²/sec (kinematic viscosity), (ii) the cardiac output of the patient, which of course varies depending upon the level of CHF and activity, and (iii) the diameter of the patient's descending aorta, which varies from patient to patient but is about 21 mm for an average adult, one can determine the flow rate Q that would result in a velocity through the aorta necessary to attain a Reynolds number of at least 2300 at its peak during the patient's heart cycle. Based upon that determination of Q, one may adjust the output of the pump of the present invention to attain the desired turbulent flow characteristic through the aorta, enhancing mixing of the blood therethrough.

One may use ultrasound (e.g., echocardiography or abdominal ultrasound) to measure the diameter of the aorta, which is relatively uniform in diameter from its root to the abdominal portion of the descending aorta. Furthermore, one may measure cardiac output using a thermodilution catheter or other techniques known to those of skill in the art. Finally, one may measure viscosity of the patient's blood by using known methods; for example, using a capillary viscosimeter. It is expected that in many cases, the application of this embodiment of the present method will provide a basis to more finely tune the system to more optimally operate the system to the patient's benefit. Other methods contemplated by the present invention may include steps to assess other patient parameters that enable a person of ordinary skill in the art to optimize the present system to ensure adequate mixing within the vascular system of the patient.

Alternative inventive methods that provide the benefits discussed herein include the steps of, prior to applying a shape change therapy, applying a blood supplementation system (such as one of the many examples described herein) to a patient, whereby the methods are designed to improve the ability to reduce the size and/or wall stress of the left ventricle, or both ventricles, thus reducing ventricular loading. Specifically, one example of such a method comprises the steps of providing a pump configured to pump blood at subcardiac rates, providing inflow and outflow conduits configured to fluidly communicate with non-primary blood vessels, fluidly coupling the inflow conduit to a non-primary blood vessel, fluidly coupling the outflow conduit to the same or different (primary or non-primary) blood vessel and operating the subcardiac pump in a manner, as described herein, to reduce the load on the heart, wherein the fluidly coupling steps may comprise anastomosis, percutaneous cannulazation, positioning the distal end of one or both conduits within the desired terminal blood vessel or any combination thereof. The method further comprises, after sufficient reduction in ventricular loading, applying a shape change therapy in the form of, for example, a cardiac reshaping device, such as those referred to herein, or others serving the same or similar function, for the purpose of further reducing the size of and/or wall stress on one or more ventricles and, thus, the heart, and/or for the purpose of maintaining the patient's heart at a size sufficient to enhance recovery of the patient's heart.

### II. Blood Conduit Connectors

As discussed above, techniques and systems have been developed to treat a patient that involve coupling a blood-flow conduit or circuit with a patient's vasculature. Such systems are sometimes configured to be implantable and sometimes have subcomponents or subassemblies that are separable. FIGS. 17-49 show features that can be incorporated into a variety of blood conduit connector assemblies or connector devices. Such devices can be configured to provide a secure connection between a source of whole blood or a subset thereof and a conduit that can be coupled with a patient's vasculature. For example, the secure connection can be between a pump, e.g., an implantable pump, and a conduit for conveying blood between the pump and the vasculature. More particularly, the systems, devices, and method further described below can be used to connect any of the conduits, cannulae or catheters, or graft described hereinabove or any similar conduits, cannulae or catheters, or graft with any other component, such as a pump.

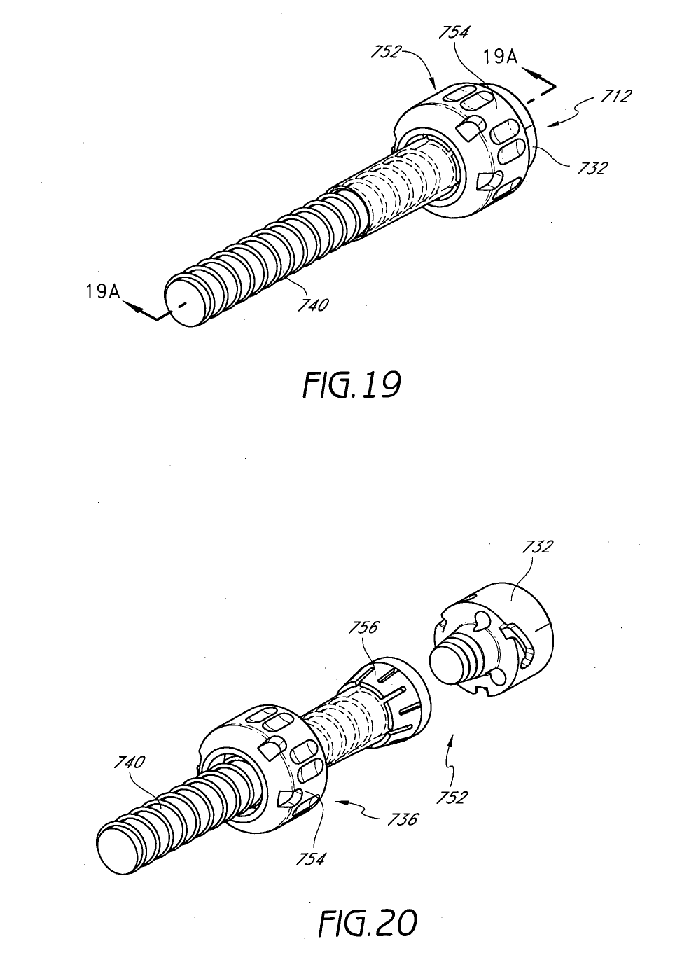

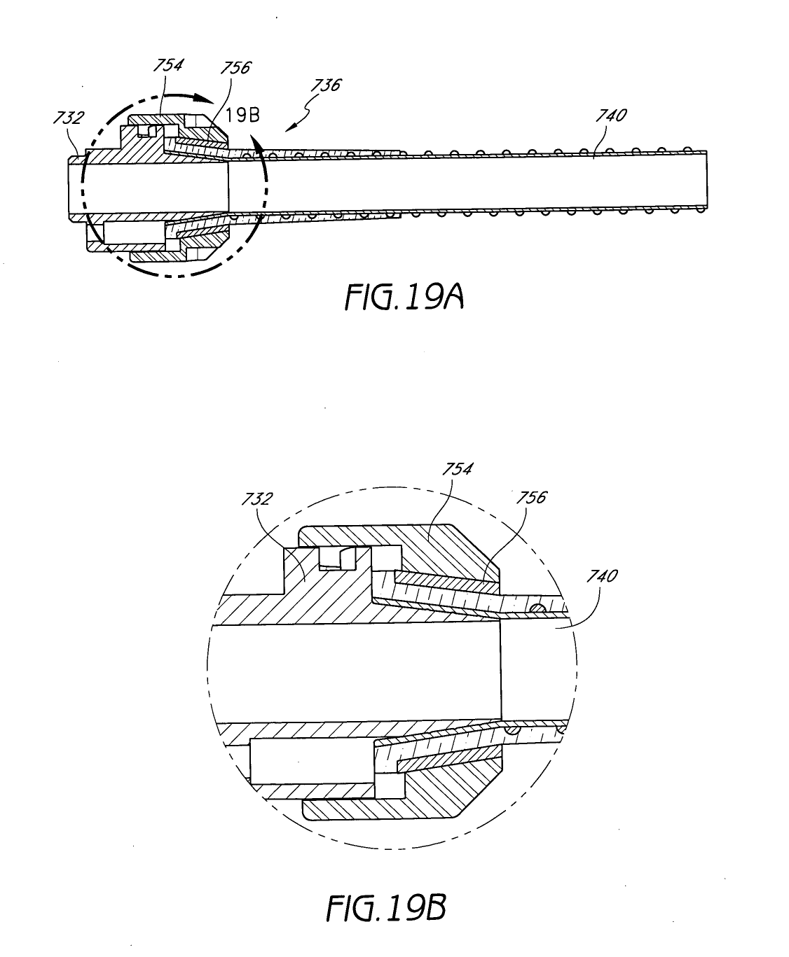

FIGS. 17-18 show one embodiment of a blood conduit connector applicator assembly 704. The blood conduit connector applicator assembly 704 includes an applicator tool 708 and a blood conduit connector 712. As discussed further below, the applicator tool 708 is adapted to engage the blood conduit connector 712 to enable a user to securely connect the blood conduit connector 712 to another structure, e.g., a pump. As discussed further below, the applicator tool 708 can be provided with a drive feature 716 that can engage a corresponding driven feature 720 of the blood conduit connector 712 so that a force can be transmitted to the blood conduit connector 712 to cause the blood conduit connector to engage another component, e.g., a pump or a pump fitting associated therewith. The blood conduit connector 712 also includes a pump fitting 732 in some embodiments, as discussed further below. In certain embodiments, the connector tool 708 can be used to connect or disconnect a graft assembly 736 from the pump fitting 732 of the connector 712 (FIGS. 19 and 20). Although the pump fitting 732 is shown as being a separate component from a pump with which the pump fitting may be coupled, the pump fitting also can be an integral part of a source of blood or pump. In other embodiments, a connector fitting can be provided that is similar to the pump fitting 732 but that forms a part of or is coupled with another components, such as another source of blood

FIGS. 19, 19A, 19B, and 20 show one embodiment of the blood conduit connector 712 in greater detail. FIGS. 19A and 19B illustrate a cut away of the blood conduit connector 712. In one form, the connector 712 includes the pump fitting 732 which can be configured to mate with the graft assembly 736.

In one embodiment, the graft assembly 736 includes a vascular graft 740 that, as discussed further below, can be configured to engage the pump fitting 732. In one embodiment, the vascular graft 740 is flared at a proximal portion. The flared proximal portion enables the graft assembly 736 to be advanced over a corresponding structure on the pump fitting 732. In one embodiment, the vascular graft 740 includes a portion, e.g., at or proximate the proximal end, that provides one or more mechanical or structural enhancements, such as a strain relief, a reinforcement, or a shape maintenance aspect. Such enhancement may be provided by a thickening of the proximal portion of the vascular graft 740 or provision of a secondary material. The secondary material can be provided to maintain the shape of the proximal section of the vascular graft 740 or to provide some other advantageous feature, as discussed further below. The secondary material can be formed or disposed about the vascular graft 740, e.g., by overmolding. The secondary material can be a polymeric material.

The graft assembly 736 can be coupled with a locking mechanism 752 that is configured to secure or to maintain the connection between the vascular graft 740 and a source of blood, such as a pump, e.g., between the vascular graft 740 and the pump fitting 732. In one embodiment, the locking mechanism 752 includes a member 756 and a coupler 754. One or both of the member 756 and the coupler 754 can operate by generating or transmitting a compression force to internally disposed structures. The coupler 754 can be a fitting in some embodiments. In one arrangement, at least some of these components are formed of or comprise a biocompatible material to enable them to be implanted for several days or several months. In other arrangements, the materials are used for at least some of the components to enable them to be implanted for several months to a year or more. For example, the pump fitting 732 can be made of a biocompatible metal, such as titanium or any suitable alloy thereof. In one embodiment, all of the components of the connector 712 are implantable. For implantable systems, at least some and in some cases all of the components of the blood conduit connector 712 are formed of or comprise biocompatible materials.

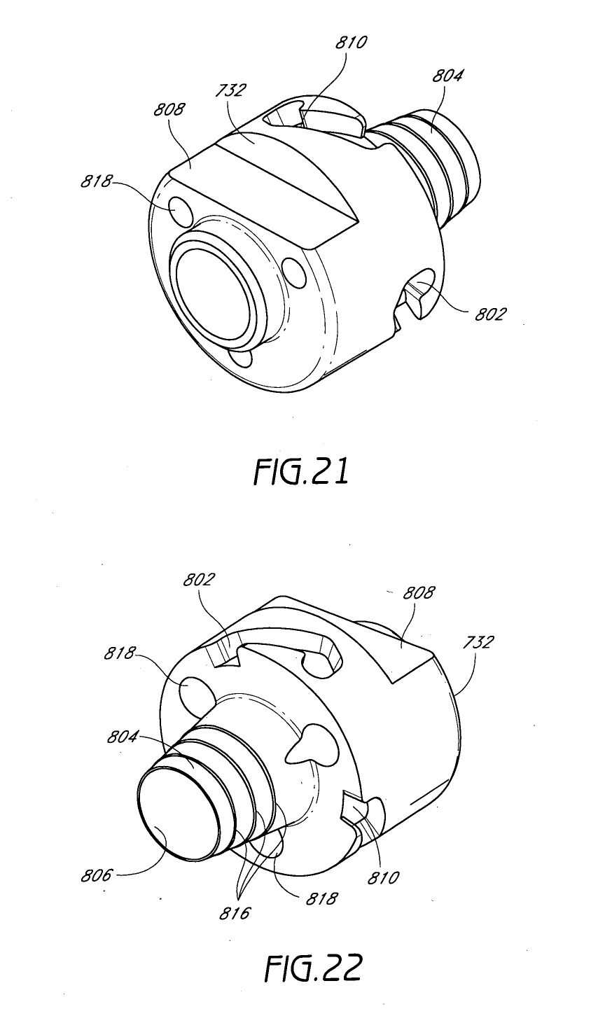

With reference to FIGS. 21-28, the pump fitting 732 includes a body 802 and a cannula interface 804. A passage 806 passes through the body 802 and the cannula interface 804. In use, the body 802 is connected to the pump and the passage 806 is in fluid communication with an inflow or outflow port thereof. The body 802 can be configured to maintain the orientation of the pump fitting 732 relative to the pump, e.g., by including an alignment feature such as one or more generally flat areas 808 configured to mate with a corresponding flat area on the pump.

The body 802 also is configured to secure the graft assembly 736, e.g., by including at least one mating feature configured to mate with the coupler 754 of the connector 712, as discussed further below. The mating features can include bayonet connections or other suitable quick connecting features. In one embodiment, a bayonet connection includes one or more, e.g., three, slots 810. The slots 810 each include a securement detent 814 and a ramped advancement portion 812 (FIG. 28) to guide motion of a mating structure, such as a pin 902 (see FIG. 39) or tab on the coupler 754 in the slot 810. As the coupler 754 is rotated relative to the pump fitting 732, the pin 902 is guided by the advancement portion 812 to move the coupler 754 towards the pump fitting 732. With continued rotation, the pin 902 reaches the securement detent 814. The movement of the pin 902 in the slot 810 as described advances the coupler 754 from a disconnected position relative to the pump fitting 732 to a connected position. In the connected position, the coupler 754 is positioned distally from a proximal-most position of the coupler 754 during travel in the advancement portion 812 between the disconnected and the connected positions. A J-shaped geometry of the slot 810 can prevent inadvertent decoupling of the coupler 754 from the pump fitting 732. Once the pin 902 is in the securement detent 814, the coupler 754 can be decoupled from the pump fitting 732 by being urged proximally, e.g., towards the pump fitting 732, and rotated such that the pin 902 or other engagement feature on the pump fitting 732 can travel through the slot 810 in the opposite direction. Thus, a bayonet connection with slots 810 allows for rapid, secure connection and disconnection without damaging the pump, cannula, or surrounding tissue.

In one embodiment, the body 802 includes three J-slots 810 that are angularly spaced evenly from one another. This configuration provides rapid attachment and release with substantially less than a complete revolution of the coupler 754, e.g., with a quarter-turn. Moreover, the bayonet connectors facilitate rapid removal and replacement of a pump or graft assembly 736 in a pumping system. In other embodiments, more or fewer J-slots 810 or advancement portions of other configurations can be used. All slots 810 have the same J-shaped configuration in the illustrated embodiment. In some pumping systems different slot and pin configurations or other engagement means can be for different pump fittings to prevent misconnections of graft assemblies to the pump. It is contemplated that other mating features, including slots having a different configuration, or mating screw threads on the coupler 754 and the body 802 can be used in other embodiments of connector 712.

Different bayonet configuration geometries can be used for inflow and outflow conduits of a pumping system to reduce the risk of misconnections. For example, an inflow pump fitting and graft assembly could have three J-slots 810 and mating pins 902 while an outflow pump fitting and graft assembly could have four J-slots 810 and mating pins 902 such that no misconnection could be made. Further, to facilitate proper connection of inflow and outflow sides of a pumping system, the pump fittings and graft assemblies can include visual cues to distinguish inflow components from outflow components such as color coding, matching marks or symbols, matching labels, or flow directional indicators.

The body 802 can also include mounting features such as at least one hole 818 therethrough to facilitate mounting of the pump fitting 732 to a pump or other structure. In the illustrated embodiment, the body 802 includes three holes 818 therethrough, angularly evenly spaced about the body. It is contemplated that in other embodiments the body could comprise more, fewer, or different locations of holes 818. In still other embodiments, the body 802 can be integrally formed with a pump or pump housing.

The cannula interface 804 can be configured as a generally elongate member extending from the body 802 and having a passage 806 therethrough. The cannula interface 804 has a relatively constant inner diameter in one embodiment. In the illustrated embodiments, the cannula interface 804 has a ramped outer surface such that the outer diameter of the tubular member is greatest adjacent the body 802.

As illustrated in FIG. 25, the cannula interface 804 can taper to a narrow edge to allow a smooth, substantially step-less or seamless transition for liquid flowing in through the passage 806 at the connection between the connection fitting 732 and the vascular graft 740. Such a transition can be advantageous as it promotes laminar flow. In applications related to conveying blood, this smooth transition for fluid flow through the connector 712, which maintains laminar flow, reduces the incidence of blood coagulation or thrombus formation.

In certain embodiments, the ramped cannula interface 804 can have at least one securement feature 816, extending from its outer surface. The securement feature 816 on the interface 804 can be a ridge, e.g., an annular ridge or barb. A combination of a ramped interface with the annular ridge(s) 816 enhances the connection between the cannula interface 804 and a conduit advanced thereover and reduces the potential for leakage from the conduit at the cannula interface 804. The combination also reduces the potential for slippage of the conduit relative to the cannula interface 804.

With reference to FIGS. 20 and 29-35, more details of the graft assembly 736 will be discussed. As illustrated in FIG. 20, the graft assembly 736 comprises a vascular graft 740, a member 756, and a coupler 754.

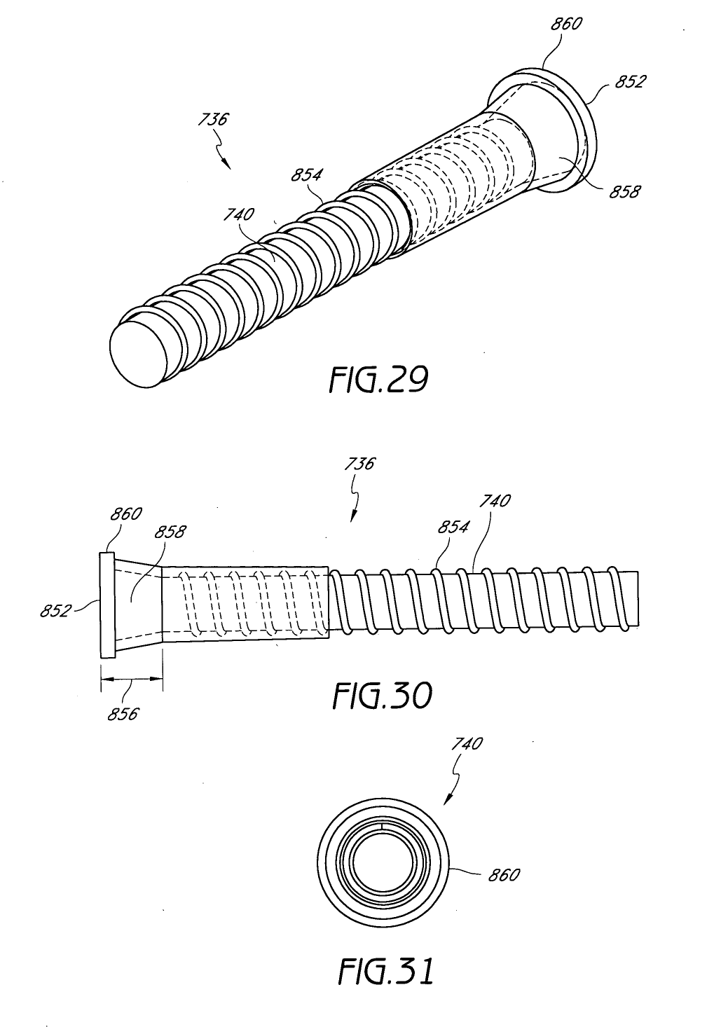

FIG. 20 shows the graft assembly 736 and the pump fitting 732 with which the graft assembly 736 mates. In this arrangement, an end of the vascular graft 740 is configured to mate with the pump fitting 732 by being flared at the proximal portion 852 of the vascular graft 740 (FIG. 30). The flared profile facilitates the advancement of the vascular graft 740 over the ramped cannula interface 804 of the pump fitting 732 because the proximal portion 852 of the vascular graft 740 is larger than a distal end of the cannula interface 804.

FIGS. 29-31 illustrate various embodiments of vascular graft 740. The vascular graft 740 has a proximal portion 852 and a lumen extending therethrough. The proximal portion 852 is flared as discussed above. In the proximal portion 852, an inner diameter of the vascular graft 740 decreases distally along a length of the vascular graft 740 over a flared portion, thus forming a flared segment 856 of the cannula 740. Distal of the flared segment 856, the inner diameter of the vascular graft 740 remains substantially constant in one embodiment. Desirably, the inner diameter of the cannula 740 distal of the flared section is approximately equal to an inner diameter of the passage 806 of the pump fitting 732. This substantial equality of inner diameters contributes to the smooth transition and substantially stepless fluid flow through the connector 712.

The flared segment 856 of the vascular graft 740 can be pre-formed. This pre-forming forms a vascular graft 740 having a flared portion in its free state, that is, before an initial advancement over the pump fitting 732. In some cases, the vascular graft 740 also maintains the flared configuration after the vascular graft 740 is disconnected from the pump fitting 732. Advantageously, this pre-formed flared segment 856 facilitates the coupling of the vascular graft 740 to the pump fitting 732. For example, the vascular graft 740 does not need to be stretched on initial advancement over the distal end of the elongate member of the pump fitting 732. Thus, the pre-formed flared segment 856 contributes to a faster connection operation. Moreover, the pre-formed flared segment 856 reduce the incidence of graft breakage from overstretching during insertion as the vascular graft 740 does not need to be stretched on initial advancement over the pump fitting 732.

The flared segment 856 of the vascular graft 740 can be formed by the insertion of a mandrel having a desired flared profile into a lumen of the graft 740. Heat can be applied to the vascular graft 740 to cause the graft to conform to the shape of the mandrel. The mandrel is then removed and the vascular graft segment allowed to cool. In some embodiments, a wall thickness of the vascular graft 740 is substantially uniform for both the flared segment 856 and distal the flared segment 856. In other embodiments the wall thickness is less toward the proximal portion 852 then toward the distal portion.

In certain embodiments, the vascular graft 740 includes a strain relief member 858. The strain relief member 858 can be disposed at the proximal portion 856 of the vascular graft assembly 740. Desirably, the strain relief member 858 allows the vascular graft 740 to withstand coupling and decoupling cycles with the pump fitting 732 without significant degradation or failure. The strain relief member 858 can prevent a pre-formed flared segment 856 of the vascular graft 740 from contracting into a non-flared state, e.g., if the graft 740 is formed of an elastic material. Additionally, the strain relief member 858 can reduce the potential for kinking of the vascular graft 740 at the connection to the pump fitting 732. In some embodiments, the strain relief member 858 is a segment that has been overmolded about the vascular graft 740. As illustrated, the overmold segment is disposed about the vascular graft 740 and extends from the proximal portion 852 distal the flared segment 856. The strain relief member 858 can be formed of silicone. In other embodiments, the strain relief member 858 may be constructed of other materials and can have a different geometric configuration for example, extending only partially about the circumference of the vascular graft 740, extending only over the flared segment or extend over only a portion of the flared segment.

In certain embodiments, the vascular graft 740 includes an anchor member 860 at the proximal end. In some embodiments, the anchor member can be a flange. The anchor member 860 decreases the likelihood that the graft assembly 736 will be pulled out of the connector 712 inadvertently, away from the pump. In some embodiments, the anchor member 860 prevents a member 756 and a coupler 754 disposed on the end of the vascular graft 740 from falling off of the graft assembly 736. In the illustrated embodiments, the anchor member 860 comprises a flange formed on the strain relief member 858. In other embodiments, the anchor member 860 can be integrally formed with the vascular graft 740. When used in conjunction with a bayonet connection including a slot 810 geometry as discussed above, the flange desirably comprises a compressible material, such as silicone, so that the coupler 754 can advance proximally farther than the connected position during connection of the coupler 754 and the pump fitting 732.

The vascular graft 740 is desirably constructed of a material that is biostable, biocompatible, and hemocompatible. Preferably, the vascular graft 740 is biocompatible for greater than 30 days when implanted. Preferably, the vascular graft 740 is biostable and resists degradation when implanted for greater than 30 days. As discussed below, however, in certain embodiments the vascular graft 740 can be designed for a specific transformation, such as gelatin absorption, in-situ. A distal portion of the vascular graft 740 can comprise, for example, an ePTFE tube. Advantageously, ePTFE material is widely available and widely used in surgical devices. Thus, medical professionals would not require much, if any, additional training in applying sutures to a distal end of the vascular graft 740. In other embodiments, where shorter implantation terms are indicated, the vascular graft 740 can be constructed of other materials suitable for such application.

In certain embodiments, the vascular graft 740 is configured to reduce the potential for embolization, e.g., in the form of intake of air into a pumping system during initial implant. The outer surface of the graft 740 can be infused or impregnated with gelatin or another bioabsorbable material to reduce the incidence of air permeation through the vascular graft 740 during initial implantation. Once the vascular graft 740 is implanted, the gelatin can be configured to be absorbed and replaced with blood. For example, in one arrangement blood can be replaced throughout the wall thickness of the vascular graft 740. This blood replacement enhances the hemocompatibility of the vascular graft 740.

can be configured to maintain a smooth, transitionless flow path, which is particularly useful in blood-flow applications. In some embodiments, this smooth flow path is maintained with a support member 854 integrated with the vascular graft 740 at least distal the flared segment 856. Desirably, the support member 854 substantially maintains the vascular graft 740 geometry, preventing the vascular graft 740 from developing local kinks or collapses. As illustrated, the support member 854 comprises a helical reinforcing rib that is extends around the vascular graft 740 distal the flared segment. The reinforcing rib can be a relatively rigid material, such as for example, a polypropylene ribbon. Other geometries and materials of reinforcing members, such as spaced annular rings or interwoven fiber matrices can be used in other embodiments of the vascular graft 740.

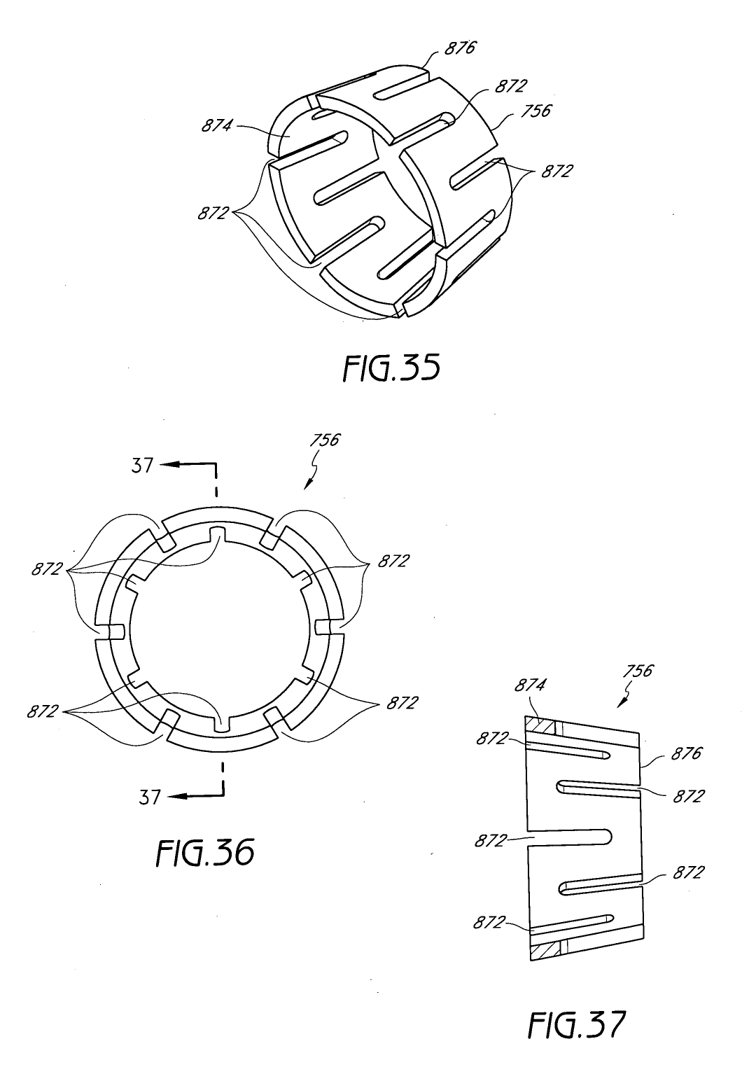

FIGS. 35-37 illustrate a member 756 configured to be disposed around the proximal portion 852 of the vascular graft. In the illustrated embodiment, the member 756 is a compression collet configured to be disposed on the vascular graft 740. The member 756 has a ramped profile, with a larger inner diameter at a proximal end 874 than at a distal end 876 such that the member 756 is configured to overlie the flared segment 856 (FIG. 30) of the vascular graft 740 and the pump fitting 732.

The member 756 has a plurality of slits 872 in one embodiment. In the illustrated embodiment, the slits 872 are arranged in an alternating fashion with one slit extending distally from a proximal end 874 adjacent to a slit extending proximally from a distal end 876 of the member 756. The slits 872 enhance the flexibility of the member 756 and the ability of the member 756 transmit substantially radially uniform loads.

During a coupling operation of the connector 712, the vascular graft 740 is advanced over the pump fitting 732 and the member 756 is advanced to the flared segment 856 of the vascular graft 740. The member 756 is configured to transmit forces and pressures to the graft substantially radially evenly such that the vascular graft 740 is securely held to the pump fitting 732.

As discussed above, the proximal end 874 of the member 756 can be configured to bear upon the anchor member 860 of the vascular graft 740. Contact between the member 756 and the vascular graft 740 prevents the vascular graft 740 from being inadvertently disconnected from the pump fitting 732.

In certain embodiments, the member 756 is constructed of a biocompatible material. Desirably, the member 756 is constructed of a biocompatible material, such as a polyetheretherketone, sometimes referred to as “PEEK”, into which the slits 872 are formed, e.g., machined. Other biocompatible materials, such titanium, could also be used for or incorporated into the member 756.

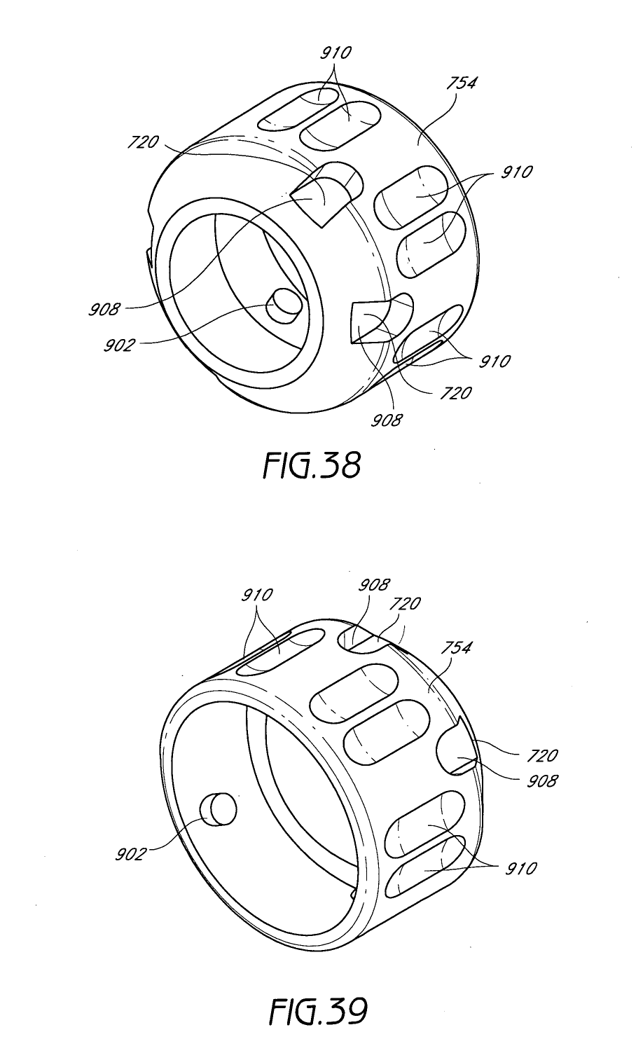

The coupler 754 will be discussed in greater detail below with reference to FIGS. 38-44. In the illustrated embodiments, the coupler 754 is a lock ring or nut configured to be disposed over the flared segment 856 of the vascular graft 740 and the member 756.

The coupler 754 includes a compression portion 904 and a locking portion 906. The compression portion 904 of the coupler 754 has a ramped inner surface configured to overlie the flared segment 856 of the vascular graft 740 and the member 756 and configured to compress the vascular graft 740 onto the pump fitting 732 to enhance the sealing between the vascular graft 740 and the pump fitting 732. See, for example, FIG. 19A. The locking portion 906 can be substantially cylindrical and is configured to extend over the body of the pump fitting when the connector 712 is connected. The locking portion 906 includes at least one mating feature such a pin 902 that is configured to mate with the pump fitting 732.

In some embodiments, the member 756 presents an outer surface upon which the compression portion 904 of the coupler 754 acts. In other embodiments, a semi-rigid or rigid member can be integrated with the proximal portion 852 of the vascular graft 740 or on an inner surface of the coupler 754, and a connection can be made without the use of a member 756. For example, a rigid polymer or metal member configured to be retained by the coupler 754 can be integrated into the vascular graft 740.

In the illustrated embodiment, the pins 902 of the coupler 754 and the slots 810 of the pump fitting 732 form a bayonet connection, allowing a user to easily and securely attach and remove the vascular graft 740 from the pump fitting 732. In use, the vascular graft 740 is advanced onto a pump fitting 732. The member 756 is advanced towards the proximal portion 852 of vascular graft 740 to overly the pump fitting 732. The slots 810 of the pump fitting 732 and the pins 902 of the coupler 754 are engaged to form a secure connection therebetween.

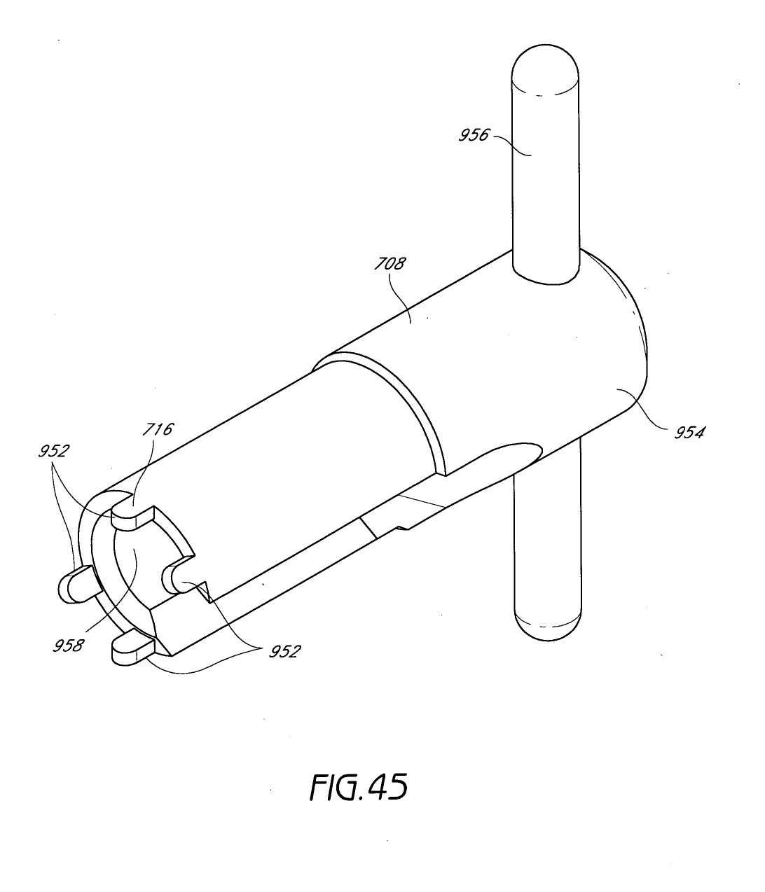

In certain embodiments, the coupler 754 can be configured to be driven by an applicator tool 708 to facilitate rapid connection and disconnection from the pump fitting 732. In some embodiments, the coupler 754 can include one or more driven features 720 (FIG. 17) positioned to correspond to drive features 716 on a applicator tool 708 (FIG. 45). In the illustrated embodiments, the driven features 720 are a plurality of recesses 908 on an exterior surface of the coupler 754. In other embodiments, the coupler 754 includes one or more ridges, grooves, depressions, lands, or other surface configurations to mate with corresponding mating features on a applicator tool 708.

In some embodiments, the coupler 754 can be configured to facilitate rapid connection and disconnection from the pump fitting 732 manually e.g., without tools. The coupler 754 can include grooves 910 or ridges on an outer surface to facilitate gripping and rotation of the coupler 754 relative to the pump fitting 732.

FIGS. 45-49 depict an applicator tool 708 for use with the connector 712 described above. Advantageously, the use of an applicator tool 708 to connect and disconnect the vascular graft 740 from the pump fitting 732 maintains sterility of the connector 712 as the connecting or disconnecting operation can be performed without directly touching the connector 712. Additionally, the use of an applicator tool 708 to connect and disconnect the vascular graft 740 from the pump fitting 732 can supply an enhanced torque to assist with connection and disconnection of potentially stuck connectors. Moreover, the use of an applicator tool 708 facilitates connection and disconnection when slippage is likely, such as, for example when the connector 712 is at least partially covered by a liquid.

The applicator tool 708 includes at least one of drive feature 716 on its distal end. The drive feature 716 can for example be a protrusion such as a tooth 952 or a lug extending from a distal end of the applicator tool 708. As illustrated in FIG. 18, a plurality of teeth 952 are positioned on the end of the connector tool 708 to mate with corresponding recesses 908 on the coupler 754. In other embodiments, the drive features 716 can be various blade, gripper, key, shaft or other structures configured to couple and decouple the coupler 754 of the connector 712.

As illustrated in FIGS. 17, 18, and 47-49, the connector tool 708 can be configured to engage the coupler 754 without substantially redirecting the vascular graft 740. In some embodiments, the connector tool 708 can include a recess 958 in an elongate tool body 954. The recess 958 can include a redirecting surface 960 to gradually shift the direction of the vascular graft 740 without forming a bend or kink in the vascular graft 740. In other embodiments, the connector tool 708 can have a body with a narrow cross-sectional profile such as a shaft with no recesses. The narrow body can be configured to pass adjacent the vascular graft 740 without substantially redirecting it.

The connector tool 708 can include a lever arm such as a grip or handle 956. The handle 956 facilitates connection and disconnection of the connector 712. The handle 956 provides a manual gripping surface for a medical professional connecting or disconnecting the connector 712. Additionally, the handle 956 provides a moment arm, and thus enhanced mechanical advantage.

In certain embodiments, a method of establishing a fluid flow connection is provided. The method comprises the steps of advancing a conduit having a pre-flared portion toward a connector fitting extending from a pump inlet port or a pump outlet port; urging a coupling device over the pre-flared portion of the conduit; and engaging the coupling device with the port. The method may, also include the step of urging a member over the flared proximal portion of the cannula.

Although the foregoing invention has been described in terms of certain preferred embodiments, other embodiments will be apparent to those of ordinary skill in the art. Additionally, other combinations, omissions, substitutions and modification will be apparent to the skilled artisan, in view of the disclosure herein. Accordingly, the present invention is not intended to be limited by the recitation of the preferred embodiments, but is instead to be defined by reference to the appended claims.

What is claimed is:

1. An apparatus, comprising:
a connector fitting having a distal end, a blood flow lumen, and an outer surface;
a conduit comprising a biocompatible material and having a pre-formed flared proximal portion;
a member configured to be disposed around and to extend along at least a portion of the proximal portion of the conduit;
a coupler configured to be urged over the member and the conduit proximally relative to the connector fitting to apply pressure to the conduit to secure the conduit to the connector fitting.

2. The apparatus of claim 1, wherein at least one of the connector fitting, the conduit, the member, and the coupler comprises a conical surface.

3. The apparatus of claim 2, wherein the connector fitting, the conduit, the member, and the coupler each comprise a conical surface

4. The apparatus of claim 3, wherein the member is configured to apply pressure to the conduit to secure the conduit to the connector fitting.

5. The apparatus of claim 1, wherein the conduit further comprises a flange at the proximal end.

6. The apparatus of claim 5, wherein the flange has a distal surface and the member has a proximal end, the proximal end of the member engaging the distal surface of the flange to couple the conduit to the connector fitting

7. The apparatus of claim 5, wherein the flange comprises a compressible material.

8. The apparatus of claim 1, wherein the conduit further comprises a strain relief member disposed over an outer surface of the conduit.

9. The apparatus of claim 8, wherein the strain relief member substantially maintains the shape of the proximal portion of the conduit.

10. The apparatus of claim 1, wherein the member comprises a collet.

11. The apparatus of claim 10, wherein the collet has a proximal end and a distal end and the collet comprises slits extending from at least one of the proximal end and the distal end.

12. The apparatus of claim 1, wherein the conduit comprises a reinforcement member.

13. The apparatus of claim 12, wherein the reinforcement member extends distally from the flared proximal portion.

14. The apparatus of claim 1, wherein the coupler comprises at least one protrusion and the connector fitting comprises at least one corresponding slot having a first end and a second end, the slot configured to receive the protrusion, and wherein movement of the protrusion in the slot from the first end of the slot to the second end of the slot corresponds to movement of the coupler relative to the connector fitting from a disconnected position to a connected position.

15. The apparatus of claim 14, wherein the slot is configured such that when the coupler is in the connected position, the at least one protrusion is positioned distally of at least a portion of the slot.

16. The apparatus of claim 1, wherein the outer surface of the connector fitting comprises at least one engagement feature configured to enhance coupling between the connector fitting and the conduit.

17. The apparatus of claim 1, wherein the blood flow lumen of the connector fitting has a first cross sectional area proximate the distal end, wherein the conduit has a lumen extending therethrough having a proximal portion and a distal portion, the distal portion having a second cross sectional area, and wherein the first cross sectional area is approximately equal to the second cross sectional area.

18. A method of establishing a connection between a conduit and a connector fitting extending from a pump inlet port or a pump outlet port, the method comprising the steps of:
advancing a conduit having a pre-flared portion toward the connector fitting;
urging a coupling device over the pre-flared portion of the conduit; and
engaging the coupling device with the connector fitting.

19. The method of claim 18, further comprising positioning a member over the flared proximal portion of the conduit between the coupling device and the port.

20. A conduit for use with a blood pump, comprising a biocompatible material and having a flared inner surface at one end thereof and configured to mechanically engage a connector.

21. The conduit of claim 20, further comprising a strain relief member disposed over an outer surface of the conduit.

22. The conduit of claim 21, wherein the flared inner surface defines a flared segment of the conduit and wherein the strain relief member extends over the flared segment.

23. The conduit of claim 22, wherein the strain relief member extends over the conduit distal the flared segment.

24. The conduit of claim 21, wherein the strain relief member comprises a biocompatible material.

25. The conduit of claim 21, wherein the strain relief member is overmolded silicone.

26. The conduit of claim 20, further comprising an anchor member positioned at the end having the flared inner surface.

27. The conduit of claim 26, wherein the anchor member is a flange.

28. The conduit of claim 26, wherein the anchor member has a distal surface configured to mechanically engage a connector.

29. The conduit of claim 20, wherein the conduit has a wall thickness and an inner diameter, and wherein an increase in the inner diameter along the flared inner surface of the conduit corresponds to a decrease in the wall thickness.

30. A system, comprising:
the conduit of claim 20; and
a member being configured to receive the conduit and to be disposed around an outer surface corresponding to the flared inner surface, the member extending along a longitudinal axis of the conduit.

31. The system of claim 30, further comprising a pump having a pump fitting comprising a blood flow lumen, and a coupler configured to be urged over the member and the conduit proximally relative to the pump fitting to apply pressure to the conduit to secure the conduit to the pump fitting.

32. The system of claim 31, wherein the pump is configured to pump blood through a patient at subcardiac volumetric rates, wherein the pump has an average flow rate between 0.1 liters/min and 3.0 liters/min.

33. The system of claim 31, wherein the pump fitting comprises a first pump fitting and the coupler comprises a first coupler, the pump further comprising a second pump fitting, and further comprising a second coupler, the first coupler configured to couple with the first pump fitting and not to couple with the second pump fitting.

34. The system of claim 31, wherein the coupler is configured to be secured to the pump fitting by an applicator tool.

35. The system of claim 34, wherein the coupler comprises at least one driven feature configured to engage at least one drive feature on the applicator tool.
